(19) United States
(12) Patent Application Publication
(10) Pub. No.: US 2019/0380628 A1
(43) Pub. Date: Dec. 19, 2019

(54) Analyte sensor apparatus and methods

(71) Applicant: GlySens Incorporated, San Diego, CA (US)

(72) Inventors: Timothy Routh, San Diego, CA (US); Michael Perkins, Los Gatos, CA (US)

(21) Appl. No.: 16/443,684

(22) Filed: Jun. 17, 2019

Related U.S. Application Data
(60) Provisional application No. 62/687,115, filed on Jun. 19, 2018.

(51) Int. Cl. A61B 5/145; A61B 5/00

(52) U.S. Cl. CPC A61B 5/14532; A61B 5/686; A61B 5/14503; A61B 5/0031

(57) ABSTRACT

Apparatus and methods for blood analyte sensing, data processing, and transmission and storage. In one embodiment, the sensor comprises a spatially compact multi-element implantable blood glucose sensor apparatus which is configured to generate signals or data relating to sensed blood glucose levels of a host being, and process the data in vivo to generate e.g., data suitable for transmission to an external receiver device for storage and indication. The implanted sensor apparatus may also determine the need for an alert. In one variant, the sensor apparatus provides for ultra-low energy consumption through a number of coordinated mechanisms, including only issuing wireless transmissions (advertisements) when communication is needed, and use of multiple “layered” operating modes. Reduced energy consumption advantageously also extends implantation longevity and reliability/availability.

### PRIORITY AND RELATED APPLICATIONS

This application claims priority to co-owned and co-pending U.S. Provisional Patent Application No. 62/687,115 filed on Jun. 19, 2018 and entitled “Analyte Sensor Apparatus and Methods,” which is incorporated herein by reference in its entirety.

This application is generally related to the subject matter of co-owned and co-pending U.S. patent application Ser. No. 13/559,475 filed Jul. 26, 2012 and entitled “Tissue Implantable Sensor With Hermetically Sealed Housing”; Ser. No. 14/982,346 filed Dec. 29, 2015 and entitled “Implantable Sensor Apparatus and Methods”; Ser. No. 15/170,571 filed Jun. 1, 2016 and entitled “Biocompatible Implantable Sensor Apparatus and Methods”; Ser. No. 15/197,104 filed Jun. 29, 2016 and entitled “Bio-adaptable Implantable Sensor Apparatus and Methods”; Ser. No. 15/359,406 filed Nov. 22, 2016 and entitled “Heterogeneous Analyte Sensor Apparatus and Methods”; Ser. No. 15/368,436 filed Dec. 2, 2016 and entitled “Analyte Sensor Receiver Apparatus and Methods”; Ser. No. 15/472,091 filed Mar. 28, 2017 and entitled “Analyte Sensor User Interface Apparatus and Methods”; Ser. No. 15/645,913 filed Jul. 10, 2017 and entitled “Analyte Sensor Data Evaluation and Error Reduction Apparatus and Methods”; Ser. No. 15/853,574 filed on Dec. 22, 2017 and entitled “Analyte Sensor and Medicant Delivery Data Evaluation and Error Reduction Apparatus and Methods”; and Ser. No. 16/233,536 filed Dec. 27, 2018 and entitled “Apparatus and Methods for Analyte Sensor Mismatch Correction,” each of the foregoing incorporated herein by reference in its entirety.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

Moreover, the Figures herein are either © Copyright 2018-2019 GlySens Incorporated (all rights reserved), or © Copyright of their respective copyright holders.

### 1. Technical Field

The disclosure relates generally to the field of implantable analyte detection sensors and related apparatus, and analysis and processing data generated by such sensor.

### 2. Description of Related Technology

Implantable electronics is a rapidly expanding discipline within the medical arts. Owing in part to significant advances in electronics and wireless technology integration, miniaturization, performance, and material biocompatibility, sensors or other types of electronics which once were beyond the realm of reasonable use within a living subject (i.e., in vivo) can now be surgically implanted within such subjects with minimal effect on the recipient subject, and in fact convey many inherent benefits.

One particular area of note relates to blood analyte monitoring for subjects, such as for example glucose monitoring for those with so-called “type 1” or “type 2” diabetes. As is well known, regulation of blood glucose is impaired in people with diabetes by: (1) the inability of the pancreas to adequately produce the glucose-regulating hormone insulin; (2) the insensitivity of various tissues that use insulin to take up glucose; or (3) a combination of both of these phenomena. Safe and effective correction of this dysregulation requires blood glucose monitoring.

Currently, glucose monitoring in the diabetic population is based largely on collecting blood by “fingersticking” and determining its glucose concentration by conventional assay. This procedure has several disadvantages, including: (1) the discomfort associated with the procedure, which should be performed repeatedly each day; (2) the near impossibility of sufficiently frequent sampling (some blood glucose excursions require sampling every 20 minutes, or more frequently, to accurately treat); and (3) the requirement that the user initiate blood collection, which precludes warning strategies that rely on automatic early detection. Using the extant fingersticking procedure, the frequent sampling regimen that would be most medically beneficial cannot be realistically expected of even the most committed patients, and automatic sampling, which would be especially useful during periods of sleep, is not available.

Implantable glucose sensors (e.g., continuous glucose monitoring sensors) have long been considered as an alternative to intermittent monitoring of blood glucose levels by the fingerstick method of sample collection. These devices may be fully implanted, where all components of the system reside within the body and there are no through-the-skin (i.e. percutaneous) elements, or they may be partially implanted, where certain components reside within the body but are physically connected to additional components external to the body via one or more percutaneous elements. Further, such devices provide users a great deal of freedom from potentially painful fingersticking methods, as well as having to remember and take self-administered blood analyte readings.

Ideally, yet further improved implantable blood glucose and analyte sensors may provide added operational flexibility (including the degree of autonomy of operation of the implanted device with respect to any external receivers or other data receiving devices), implantation duration and reliability, enhanced wireless communication with external user devices (e.g., commodity devices such as a user's smartphone, smartwatch, sports monitoring device, or similar) for enhanced ubiquity and compatibility, and/or ease of user operation and extraction/utilization of useful data, while also maintaining a compact and nonintrusive form factor.

### SUMMARY

The present disclosure provides, inter alia, improved apparatus (including an implanted sensor used in combination with one or more external devices) and methods, for accurately providing information relating to sensed analyte data on according to, in one variant, an opportunistic communication strategy, which enables extension of battery life, extension of implantation duration, and increased freedom for the user.

In a first aspect, a blood analyte sensor apparatus is disclosed. In one embodiment, the apparatus includes a wireless interface; data processing apparatus configured for signal communication with one or more sensing elements, and for data communication with the wireless interface; data storage apparatus in data communication with the data processing apparatus; and a power source configured to provide power to at least the wireless interface and the data processor apparatus. In one variant, the data storage apparatus comprises a computer program which, when executed by the data processing apparatus, causes the sensor apparatus to: (i) receive blood analyte signals from the one or more sensing elements; (ii) process the received blood analyte signals to compute at least data indicative of a blood analyte value; (iii) store the data indicative of the blood analyte value at the data storage apparatus; and, (iv) cause periodic transmission of one or more signals via the wireless interface.

In another variant, the computer program, when executed by the data processing apparatus, further causes the sensor apparatus to: (i) based on the computed blood analyte value, determine whether a blood analyte alert condition is present; (ii) based at least in part on a determination that the alert condition is present, generate the one or more signals to comprise data indicative of the alert condition; and, (iii) based at least in part on a determination that the alert condition is not present, generate the one or more signals to comprise data indicative of no alert condition.

In one implementation, the wireless interface is configured for data communication with an external receiver apparatus. In such an implementation, the external receiver apparatus is configured to: (i) receive the one or more signals and identify the data indicative of the alert condition or the data indicative of no alert condition; (ii) in response to data indicative of the alert condition, establish a paired connection status with the sensor apparatus and receive a wireless data transmission from the sensor apparatus comprising at least the data indicative of the blood analyte value; and, (iii) in response to the data indicative of no alert condition, maintain a disconnected communication status with sensor apparatus.

In yet another variant, the computer program, when executed by the data processing apparatus, further causes the sensor apparatus to: (i) determine whether the data indicative of the blood analyte value comprises new data which has not been previously transmitted to an external receiving device; (ii) based at least in part on a determination that the data indicative of the blood analyte value comprises new data, generate the one or more signals to comprise data indicative of new data; and, (iii) based at least in part on a determination that the data indicative of the blood analyte value comprises no new data, generate the one or more signals to comprise data indicative of no new data.

In one implementation, the wireless interface is configured for data communication with the external receiver apparatus. In such an implementation, the external receiver apparatus is configured to: (i) receive the one or more signals and identify the data indicative of new data or the data indicative of no new data; (ii) in response to the data indicative new data, establish a paired connection with the sensor apparatus and receive a wireless data transmission from the sensor apparatus comprising at least the data indicative of the blood analyte value; and, (iii) in response to the data indicative of no new data, maintain a disconnected communication status with sensor apparatus.

In another variant, the wireless interface is configured for data communication with an external receiver apparatus, and the external receiver apparatus is configured to: (i) receive the one or more signals; (ii) identify whether data indicative of one or more of a new configuration or a new calibration is stored at a storage device of the external receiver apparatus; (iii) based at least in part on the data indicative of the new configuration or the new calibration, maintain a disconnected communication status with sensor apparatus; and, (iv) based at least in part on the receipt of the one or more signals and the data indicative of the new configuration or the new calibration, establish a paired connection status with the sensor apparatus and receive a wireless data transmission from the sensor apparatus comprising at least the data indicative of the blood analyte value.

In another embodiment, the blood sensor apparatus includes a data storage apparatus having a computer program which, when executed by the data processing apparatus, causes the sensor apparatus to: (i) receive blood analyte signals from the one or more sensing elements; (ii) process the received blood analyte signals to compute at least data indicative of a blood analyte value; (iii) store the data indicative of the blood analyte value at the data storage apparatus; (iv) cause periodic transmission of one or more signals via the wireless interface; and (v) based on enablement of communication with a receiving device, cause transmission of the data indicative of the blood analyte value to the receiving device.

In varying implementations, the receiving device comprises: (i) a reduced-form user-wearable external receiving device; (ii) a user's personal mobile device; (iii) a dedicated receiver and processor apparatus; (iv) a partially implanted medicant delivery apparatus; (v) a fully implanted medicant delivery apparatus; or (vi) a non-implanted medicant delivery apparatus.

In one variant, the wireless user interface is configured to operate according to a Bluetooth Low Energy (BLE) protocol.

In yet another aspect, a blood analyte sensor apparatus is disclosed. In another embodiment, the sensor apparatus includes a computer program which, when executed by the data processing apparatus, causes the sensor apparatus to: (i) receive blood analyte signals from the one or more sensing elements; (ii) process the received blood analyte signals to compute at least data indicative of a blood analyte value; (iii) store the data indicative of the blood analyte value at the data storage apparatus; (iv) cause periodic transmission of one or more signals via the wireless interface; (v) based on enablement of communication with an external device, receive one or more of configuration data or calibration data from the external device; and (vi) cause implementation of the received one or more of configuration data or calibration data.

In varying implementations, the external device comprises: (i) a reduced-form user-wearable external receiving device; (ii) a user's personal mobile device; (iii) a dedicated receiver and processor apparatus; or (iv) a calibration apparatus.

In one variant, the wireless user interface is configured to operate according to a blue tooth low energy (BLE) protocol.

In yet another aspect, a method of operating a blood analyte sensor is disclosed. In one embodiment, the method includes: (i) enabling and implanting the blood analyte sensor; (ii) enabling an external receiving device; (iii) collecting blood analyte signals from one or more sensor elements of the blood analyte sensor; (iv) processing the blood analyte signals to calculate at least a blood analyte value via processor apparatus of the blood analyte sensor; (v) storing the blood analyte value on a storage apparatus of the blood analyte sensor; (vi) based on the calculated blood analyte value, determining whether a blood analyte alert condition is present; (vii) periodically transmitting advertisement or “beacon” data via a wireless data communication interface of the sensor, the wireless data communication interface in data communication with the processor apparatus; and (viii) based on data indicative of meeting one or more communication criteria, enabling wireless data communication between the sensor and the external receiving device.

In one implementation, the one or more communication criteria comprise one or more of: (i) data indicative of the external receiving device having new calibration data; (ii) data indicative of the external receiving device having new configuration data; (iii) data indicative of the sensor requiring calibration; (iv) data indicative of the sensor having new blood analyte value data; or (v) data indicative of the sensor having a blood analyte alert condition.

In another implementation, the method further includes operating the blood analyte sensor in a training mode; and generating a user-specific sensor operational model. In one variant, the processing of the blood analyte data comprises application of the user-specific operation model in calculation on the blood analyte value.

In yet another implementation, the method further includes determining a temporal mismatch between a reference sensor element and a working sensor element of the one or more sensor elements; and generating a temporal correction via a temporal mismatch algorithm. In one variant, the processing of the blood analyte data comprises application of the temporal correction in calculation of the blood analyte value.

In still another aspect, a housing for an implantable sensor is disclosed.

In yet another aspect, a circuit board apparatus for an implantable sensor is disclosed.

In yet another aspect, an antenna for an implantable sensor is disclosed. In one embodiment, the antenna is substantially planar and comprises a printed or deposited set of traces configured to coincide with a transmissive material (e.g., end cap) on one end of the implantable sensor apparatus, and to operate in the 2.4 GHz ISM band.

In yet another aspect, a method of assembling an implantable sensor is disclosed.

In yet another aspect, a method of storing an assembled implanted sensor to extend battery life is disclosed.

In still another aspect, a method of operating an implanted sensor to extend battery life is disclosed.

In a further aspect, methods and apparatus for utilizing indirect wireless signal propagation paths for wireless data communication between a physiologic sensor and a computerized device are disclosed. In one variant, indigenous signal addition capabilities of a commodity wireless PAN interface are used to enable greater signal strength both at the implanted sensor and the external computerized device.

In another aspect, implantable sensor apparatus configured for, after implantation thereof, opportunistic wireless communication with an external computerized apparatus, is disclosed. In one variant, the opportunistic wireless communication is enabled by at least data processing logic of the sensor apparatus which is configured to generate and evaluate blood analyte levels, the evaluation to determine the need for wireless communication. In another variant, the opportunistic wireless communication enables reduced electrical power consumption by the sensor apparatus when implanted by at least obviation of one or more wireless communications with the external computerized apparatus.

Other features and advantages of the present disclosure will immediately be recognized by persons of ordinary skill in the art with reference to the attached drawings and detailed description of exemplary embodiments as given below.

### BRIEF DESCRIPTION OF THE DRAWINGS

FIGS. 1A-1C are top perspective, side and front views respectively of one exemplary embodiment of a fully implantable biocompatible sensor apparatus useful with various aspects of the present disclosure.

FIGS. 1D-1E are bottom and top elevation views respectively of one exemplary embodiment of a fully implantable biocompatible sensor apparatus useful with various aspects of the present disclosure.

FIGS. 1F-1H are top and bottom perspective transparent views of the sensor apparatus of FIGS. 1A-1E, showing various internal components and layout.

FIG. 11 is a bottom elevation view of one embodiment of an internal circuit board assembly of the sensor apparatus of FIGS. 1A-1E.

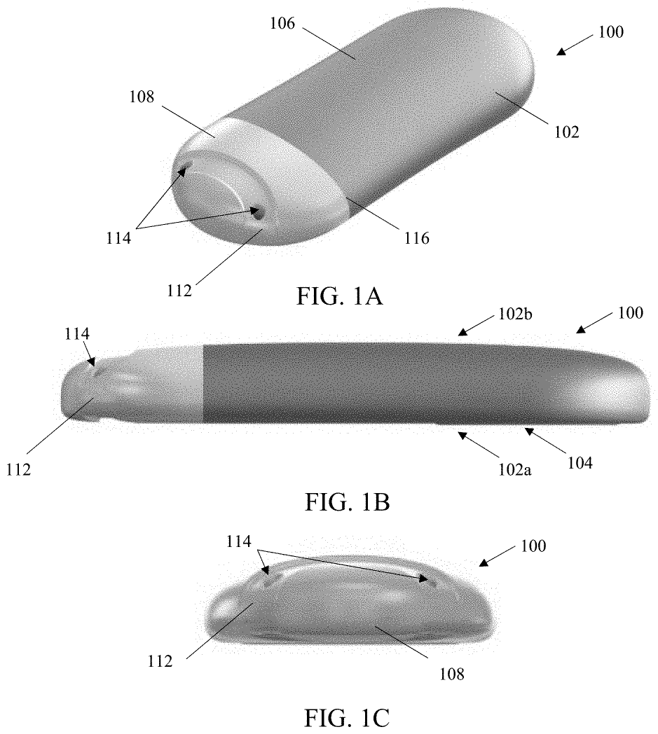

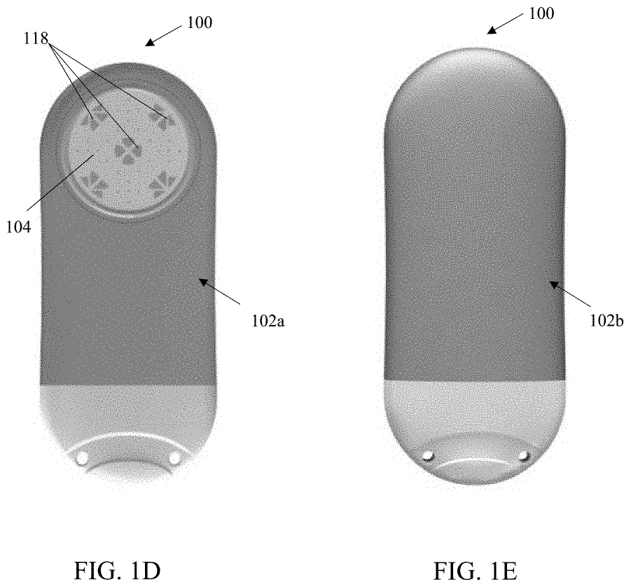

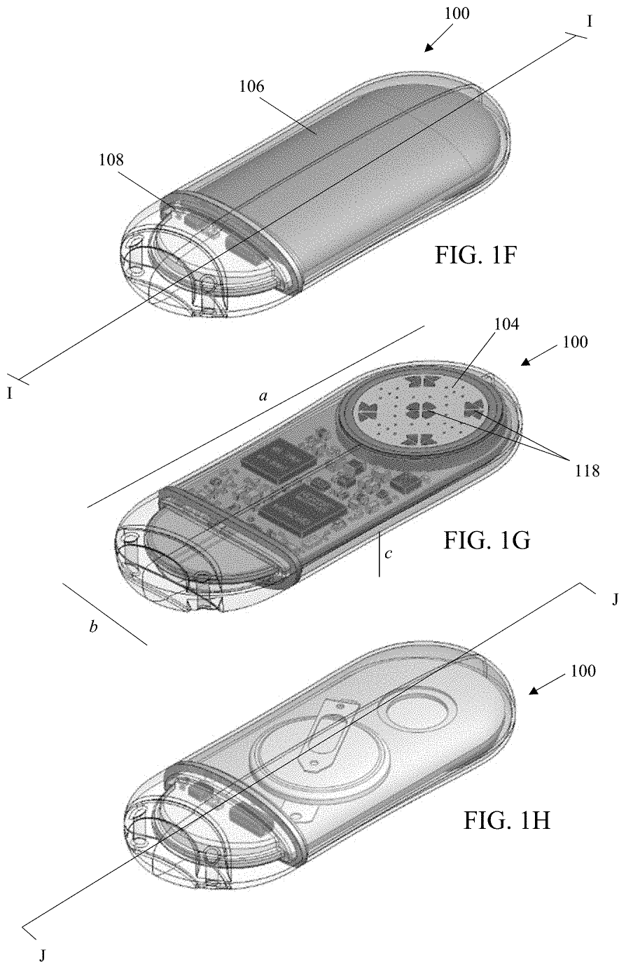

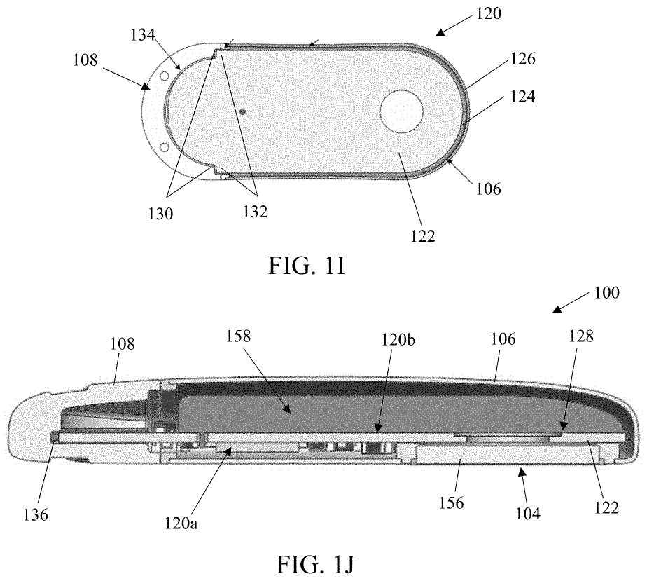

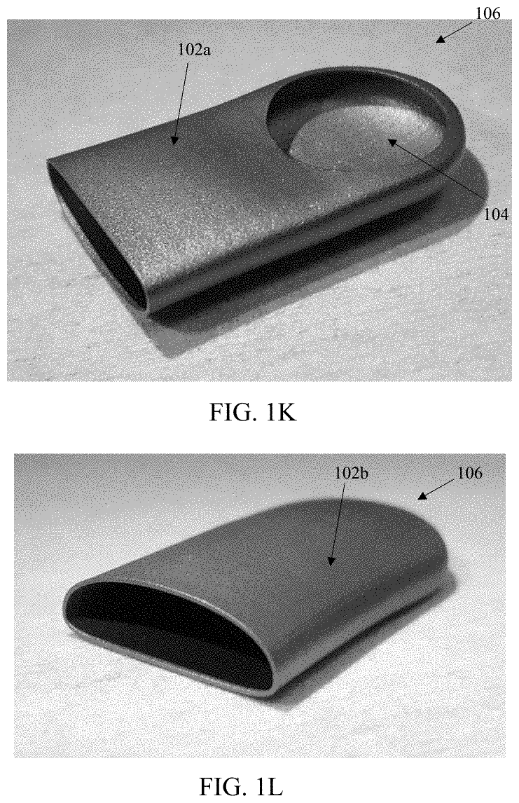

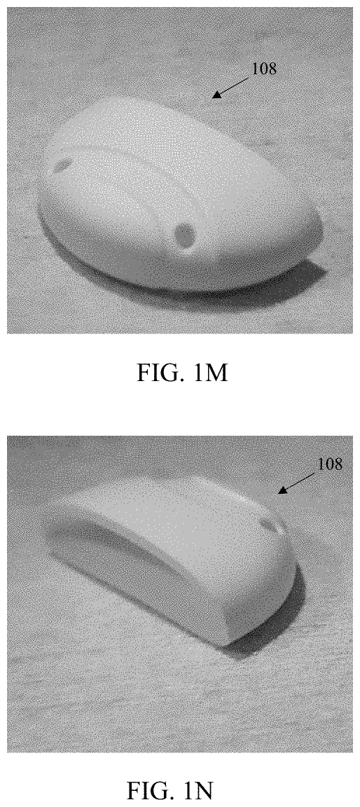

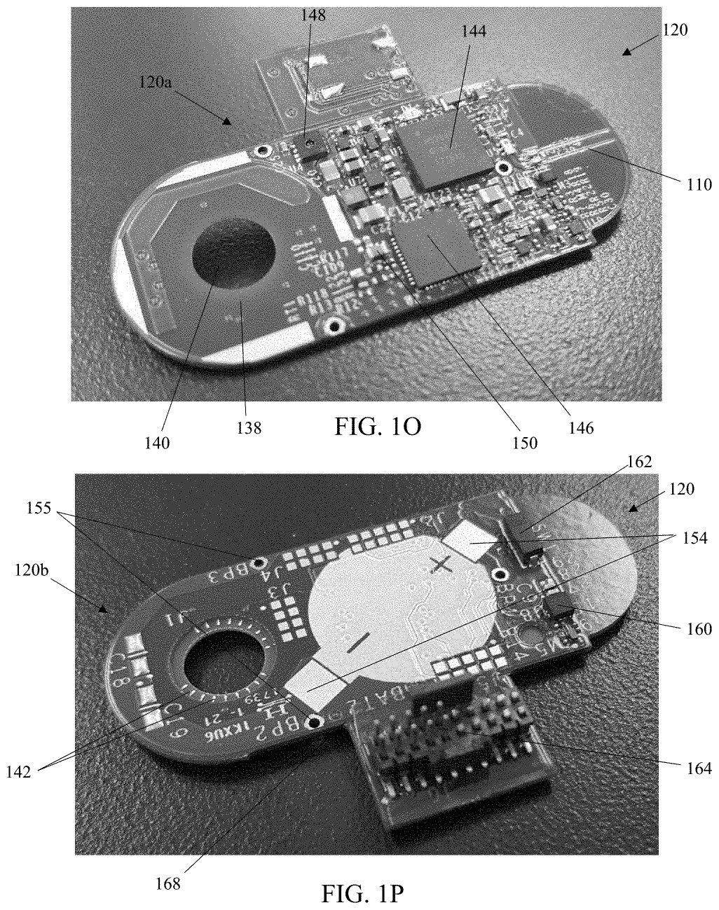

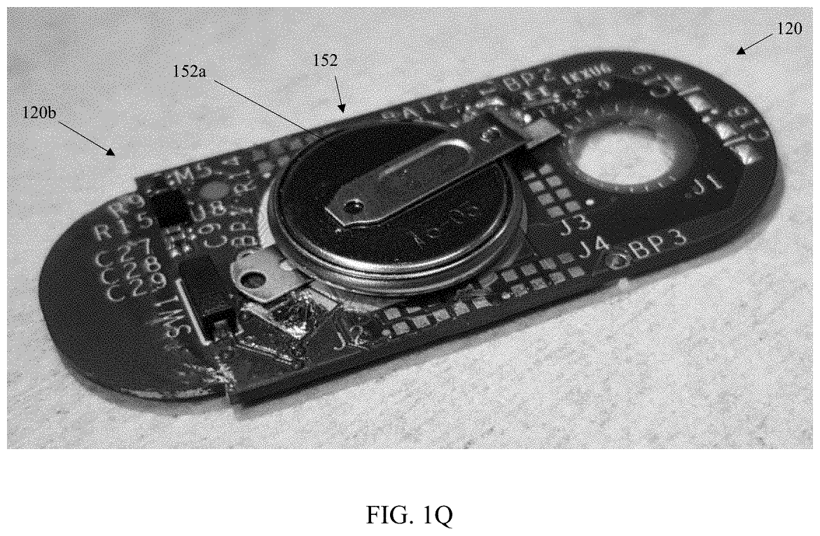

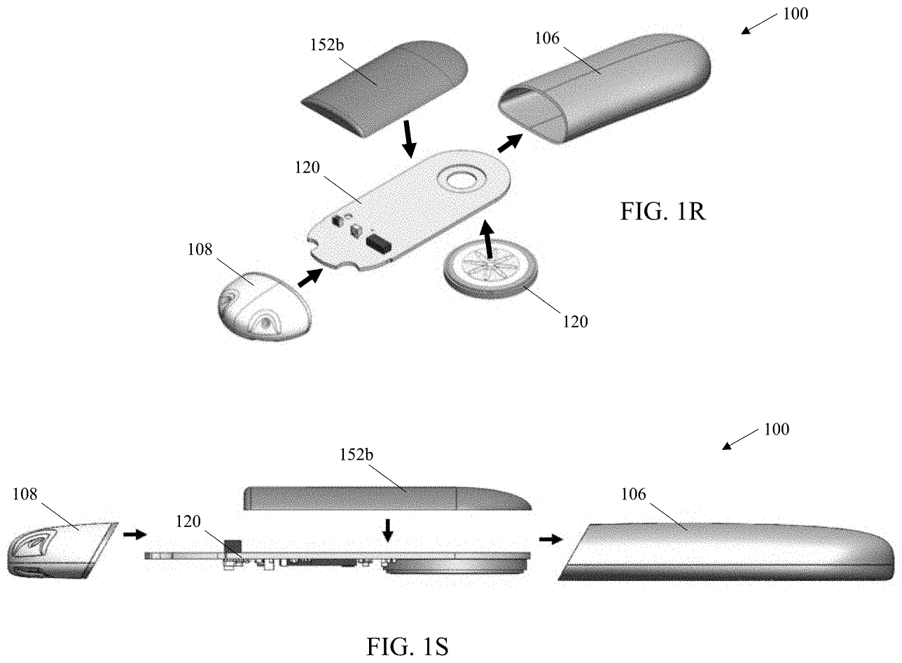

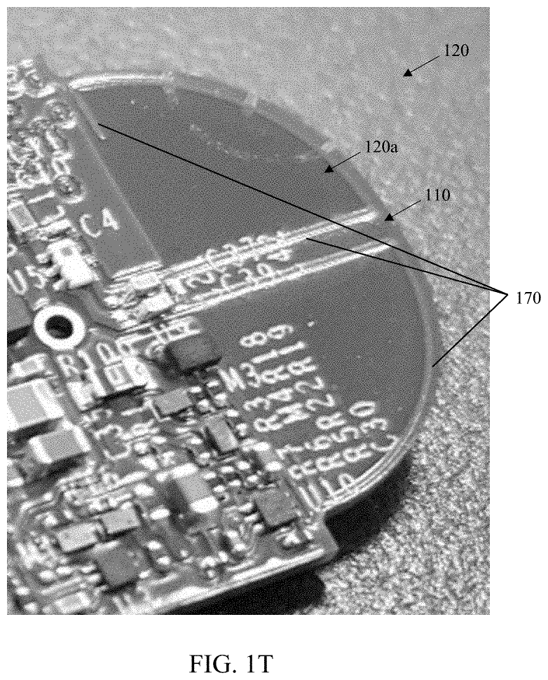

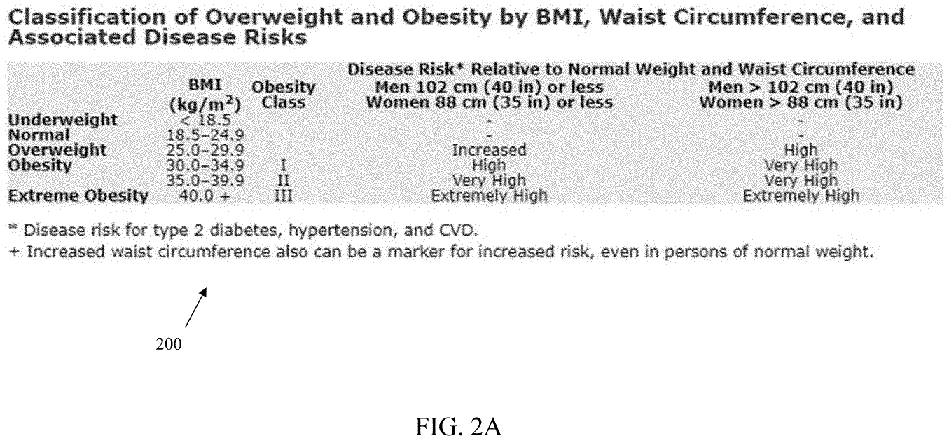

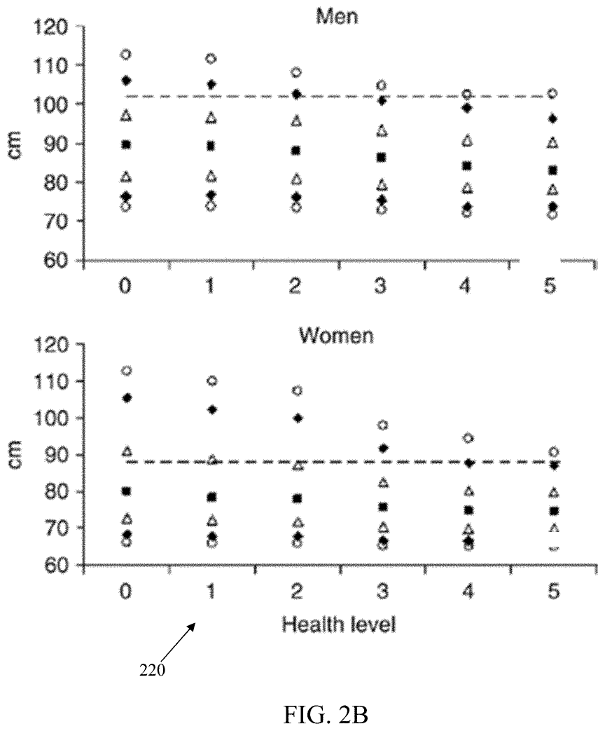

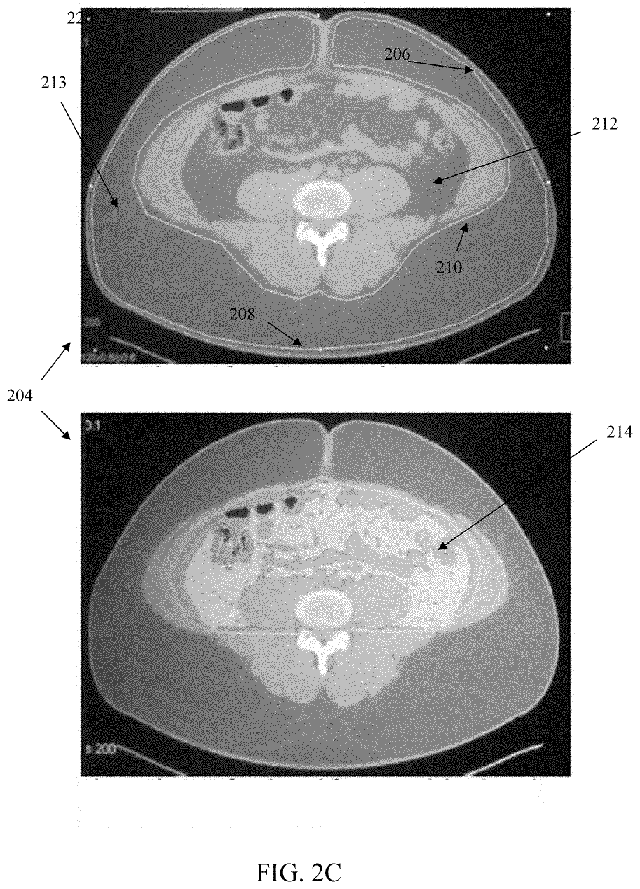

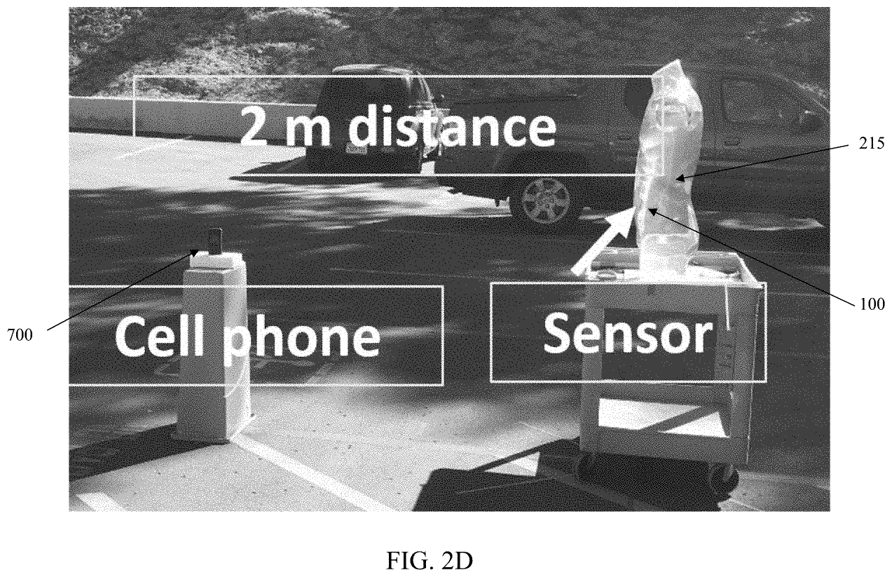

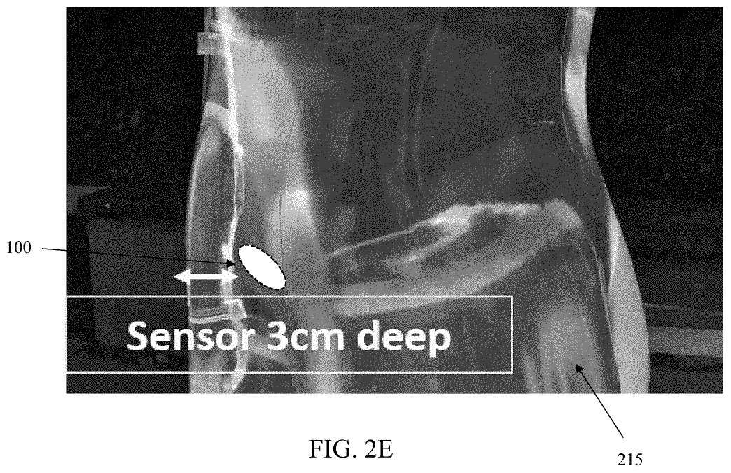

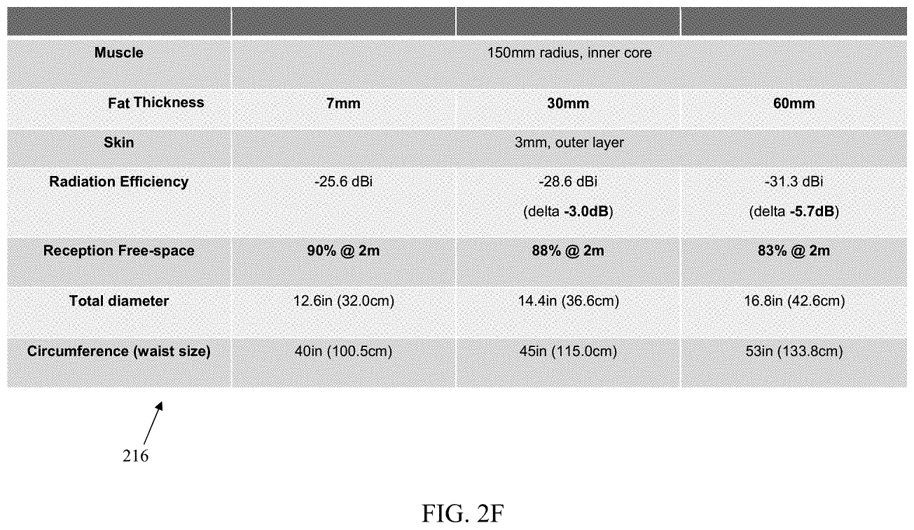

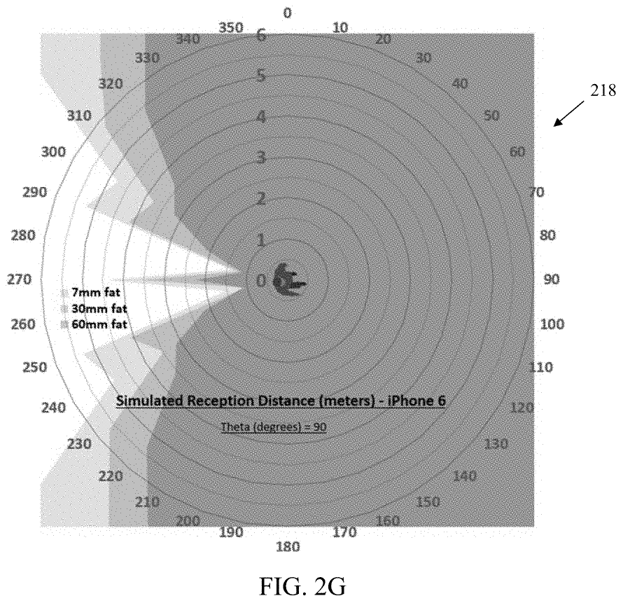

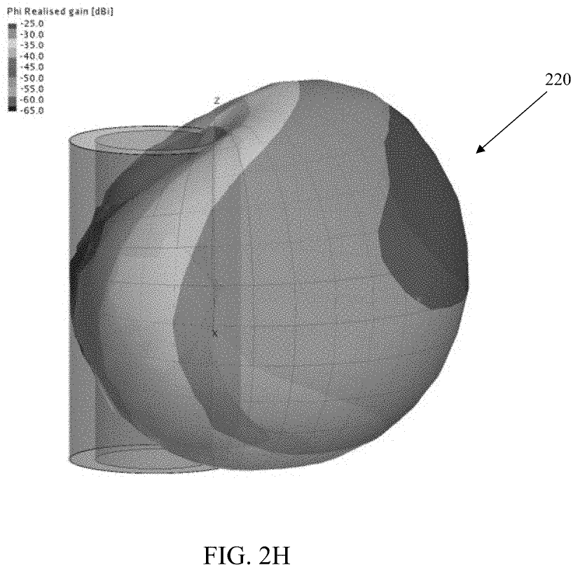

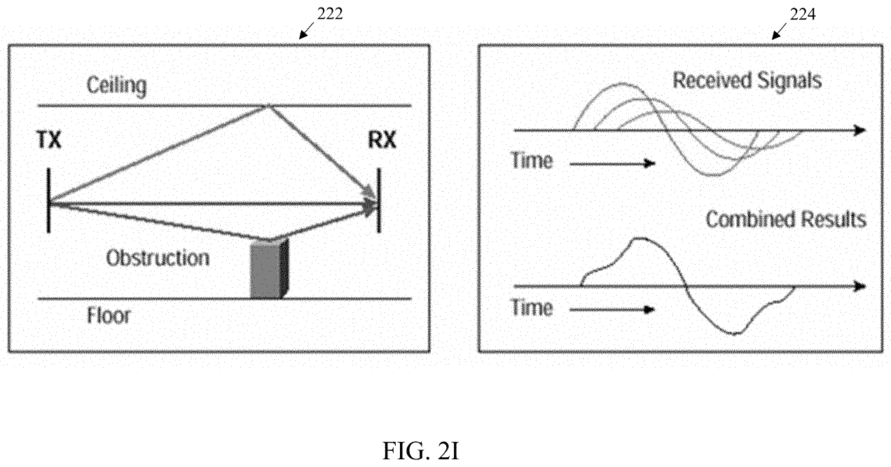

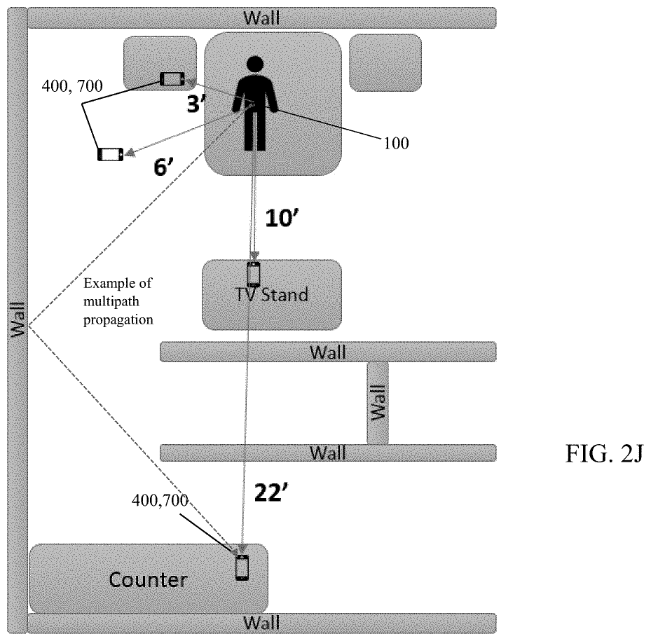

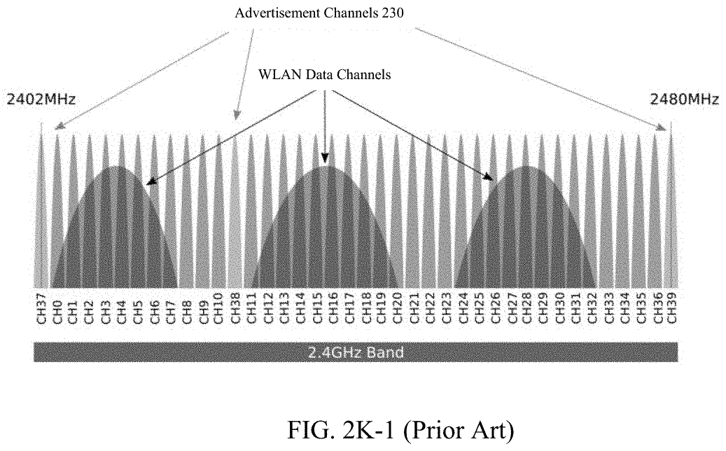

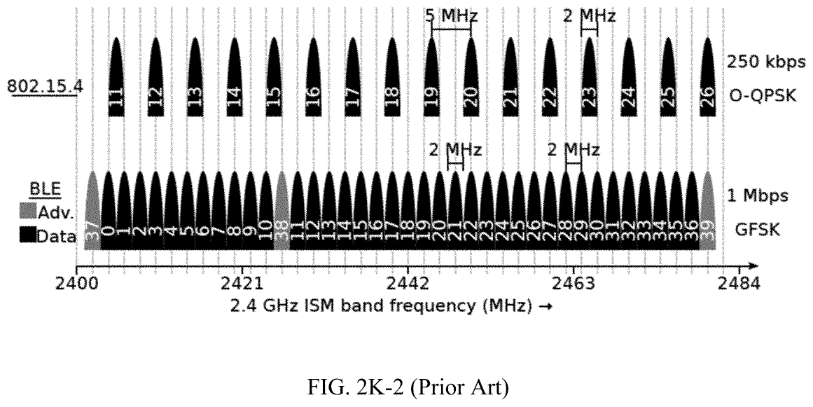

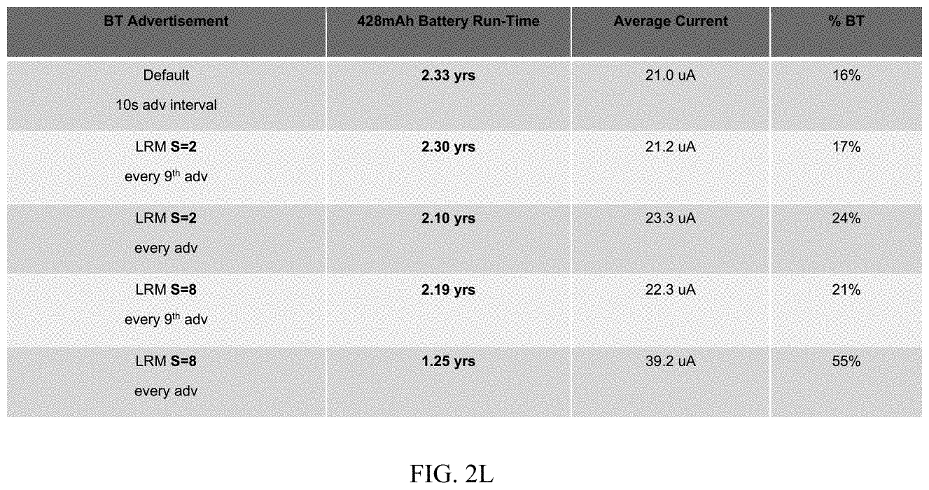

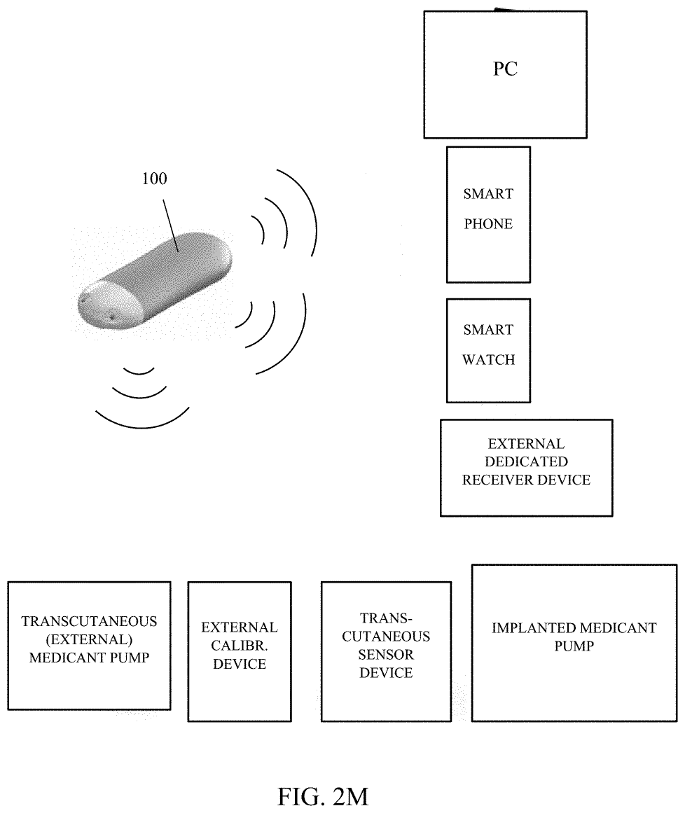

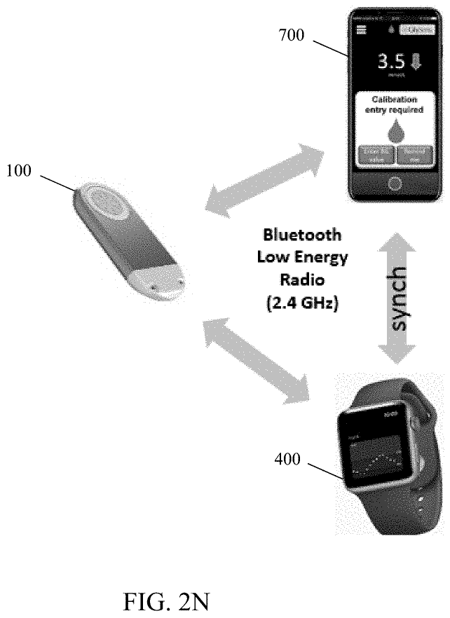

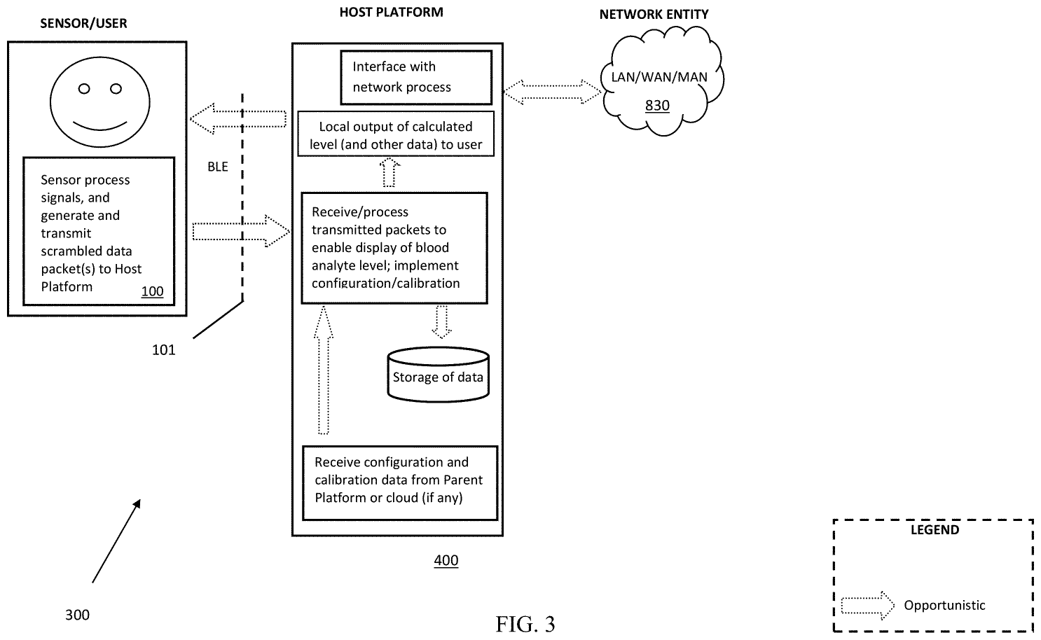

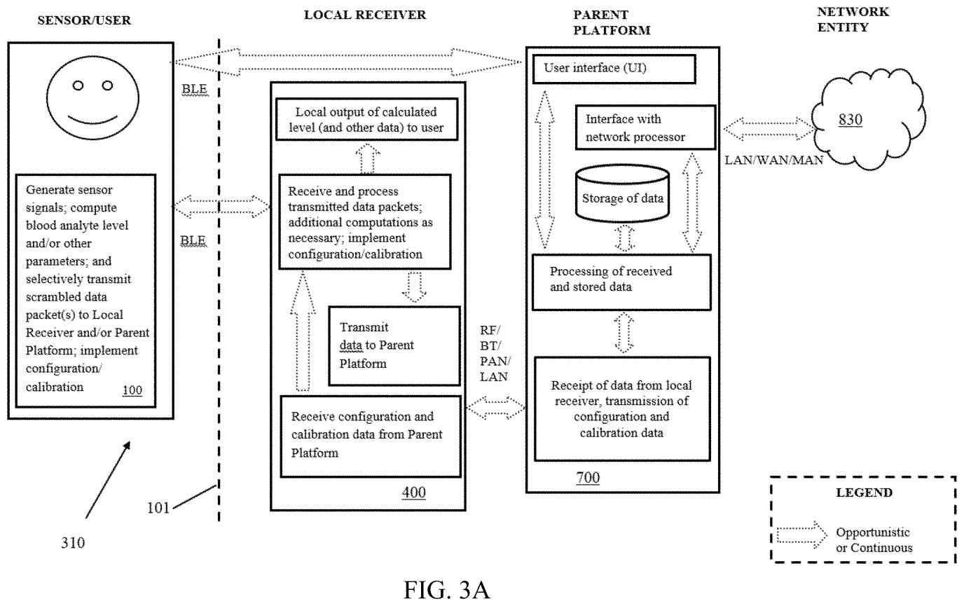

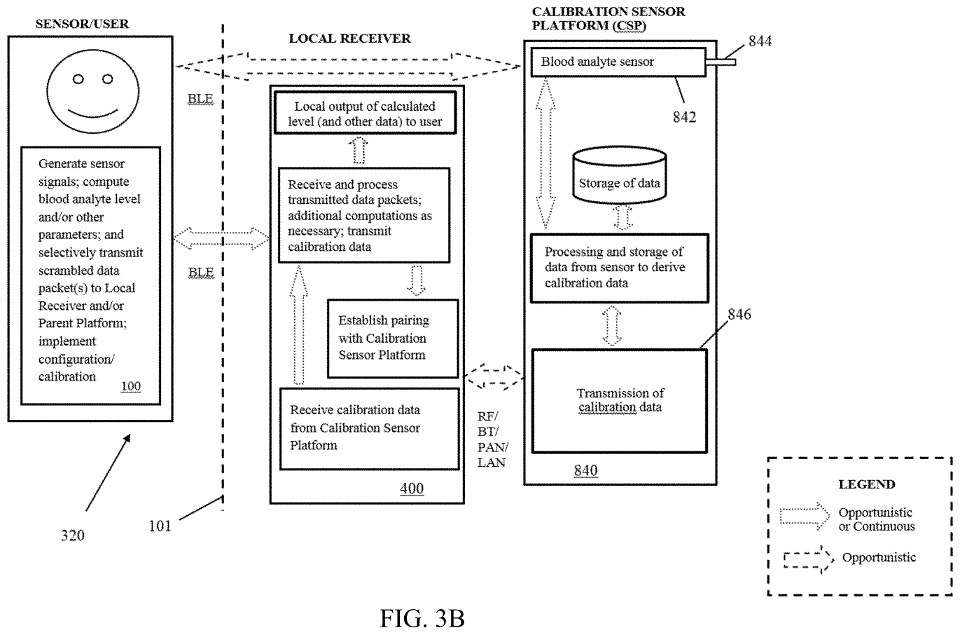

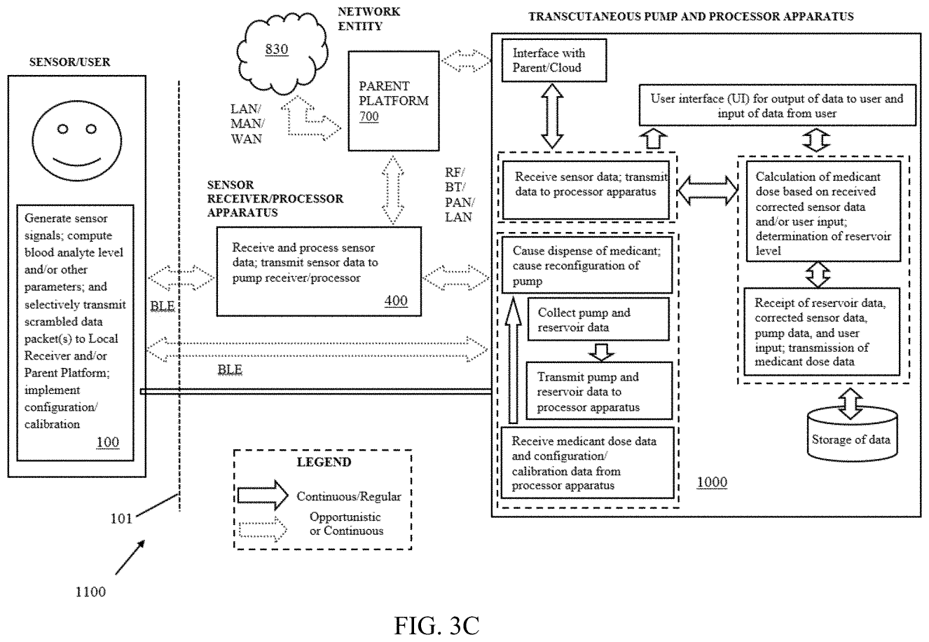

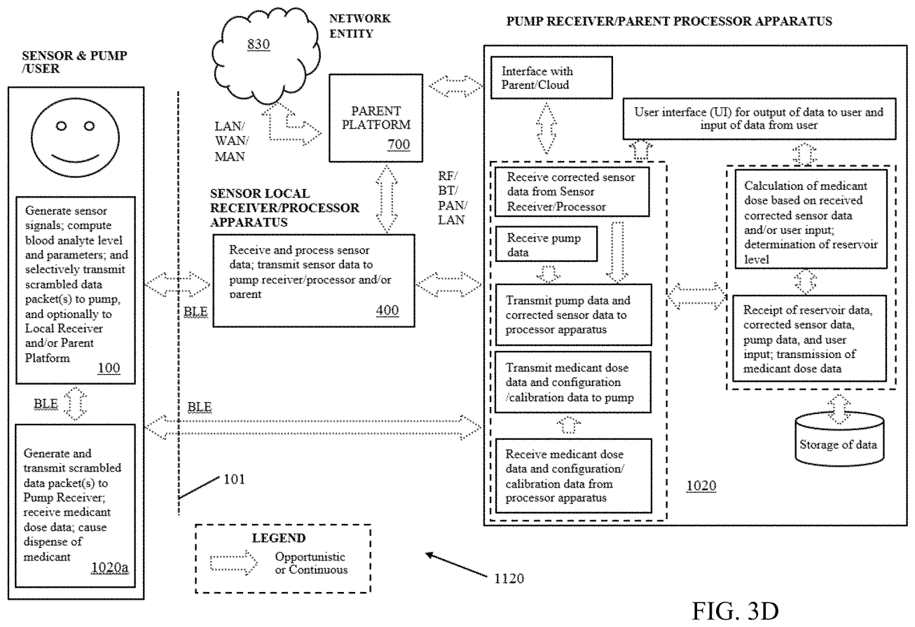

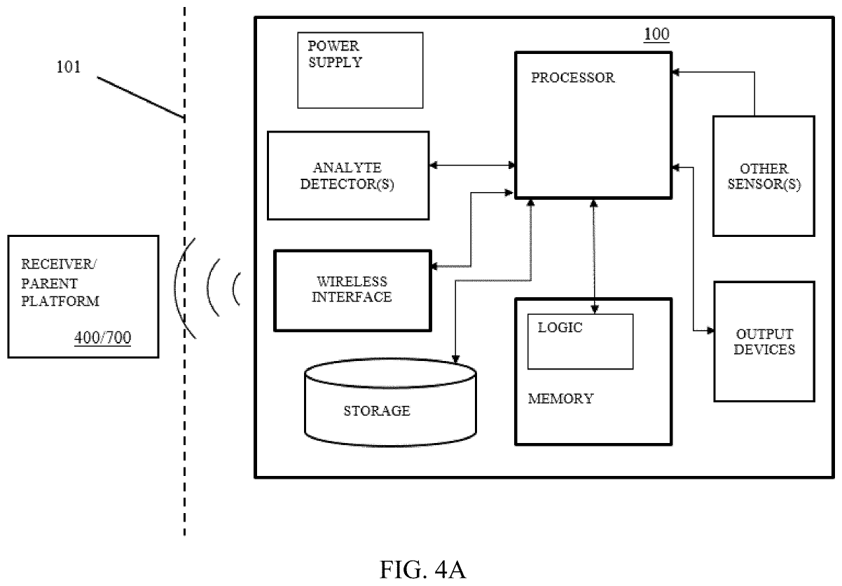

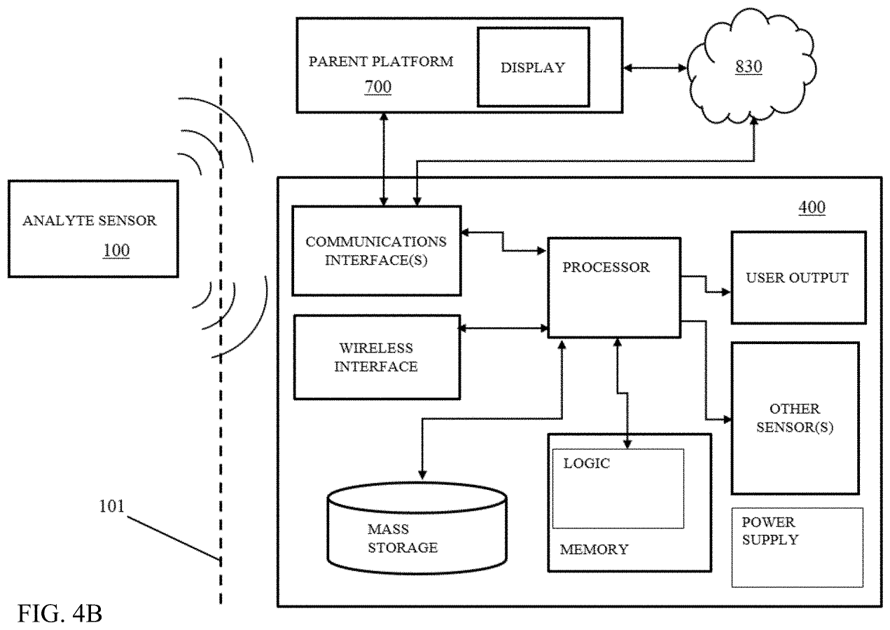

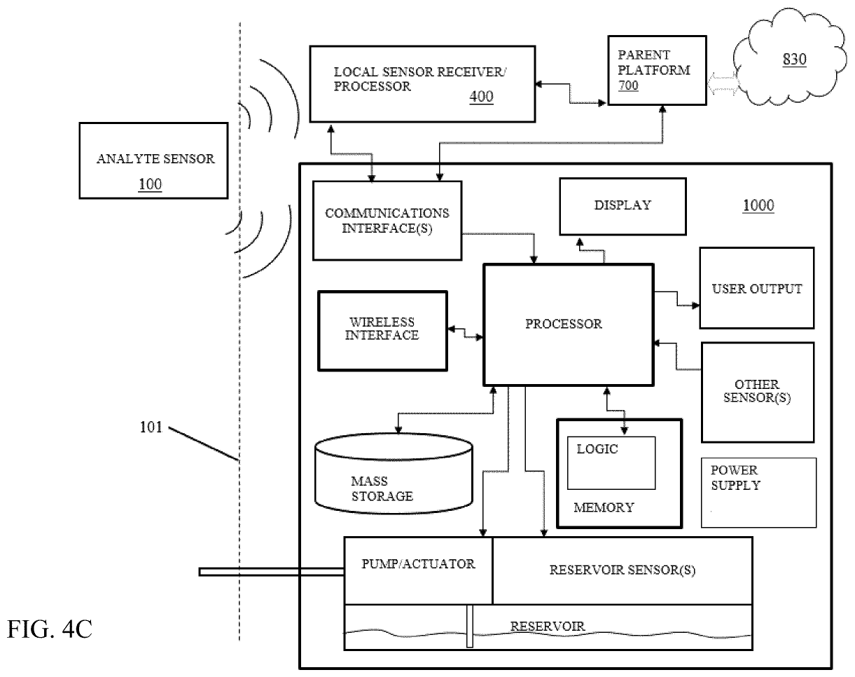

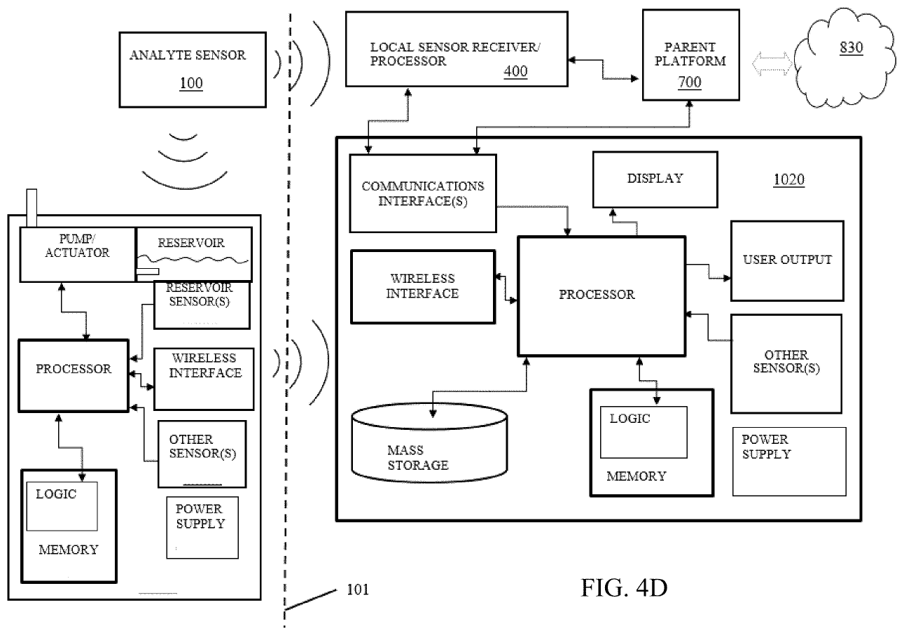

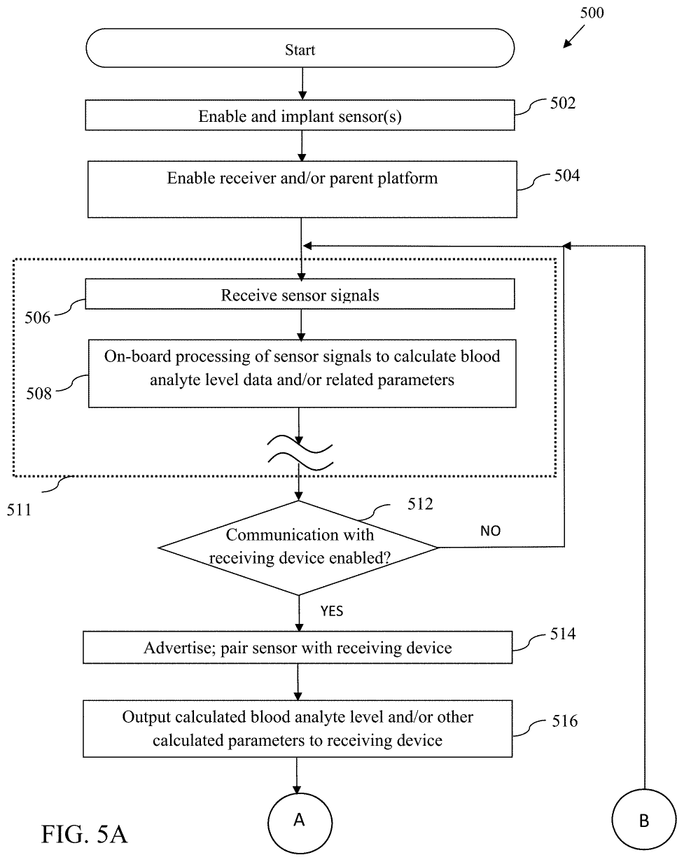

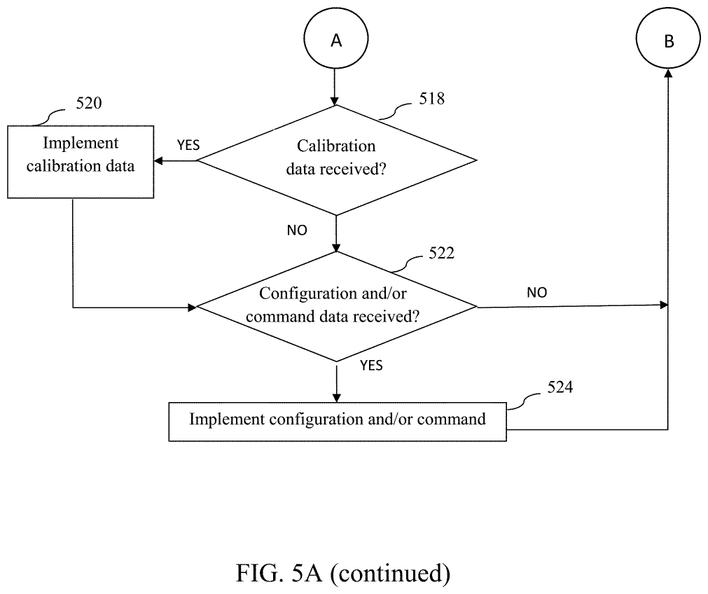

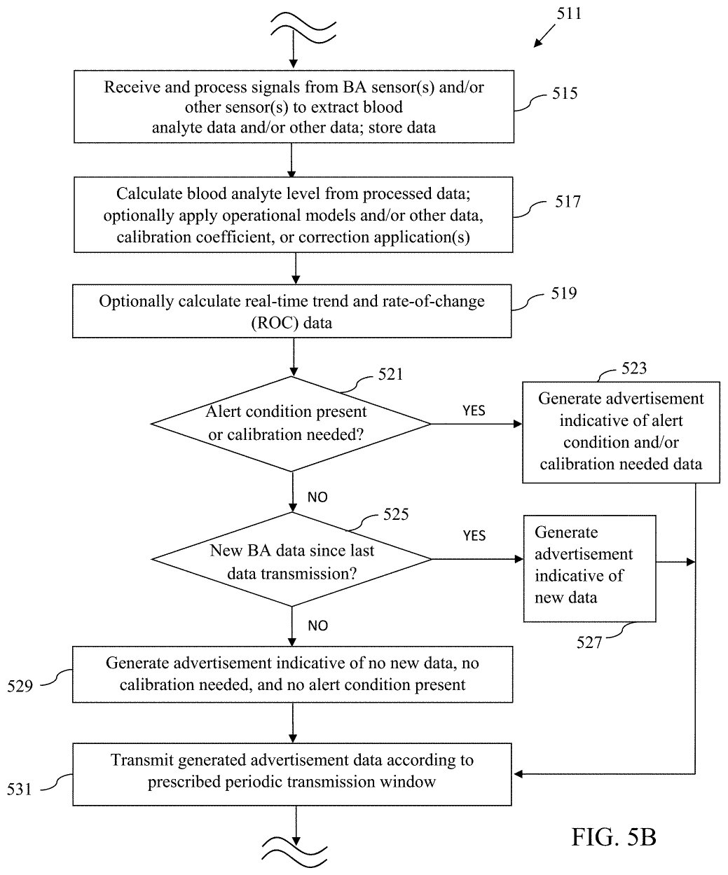

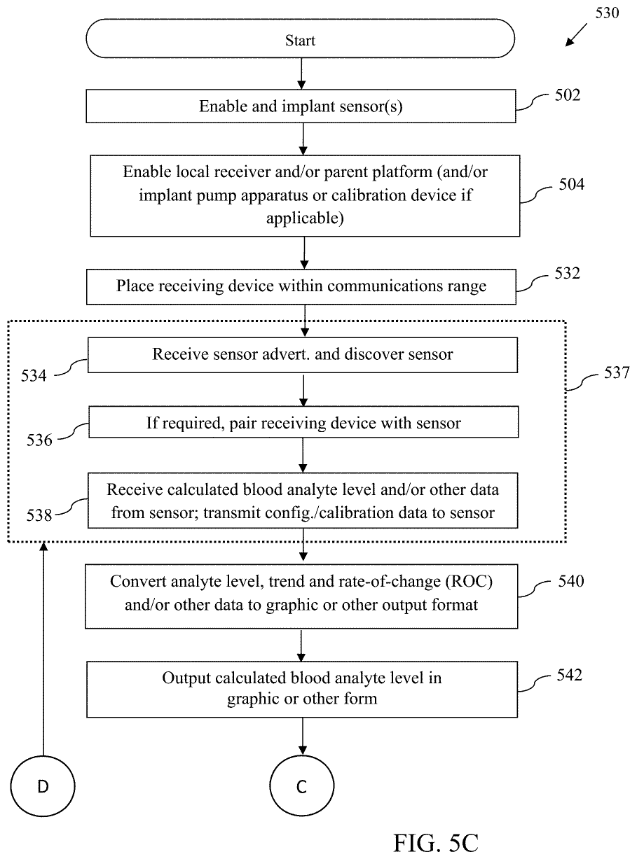

FIG. 1J is a cross-sectional view of the sensor apparatus of FIGS. 1A-1E taken along line J-J of FIG. 1H.

FIG. 1K-1L are perspective views of an outer body portion of the sensor apparatus of FIGS. 1A-1E.

FIG. 1M-1N are perspective views of an end cap body portion of the sensor apparatus of FIGS. 1A-1E.

FIG. 1O-1P are top and bottom perspective views, respectively of an interior PCB assembly of the sensor apparatus of FIGS. 1A-1E.

FIG. 1Q is a perspective view of the PCB of FIG. 1P with battery installed.

FIGS. 1R-1S are exploded views of the sensor apparatus of FIGS. 1A-1E.

FIG. 1T is a perspective view of the PCB of FIG. 1O showing the exemplary antenna configuration in detail.

FIGS. 2A and 2B are tables respectively showing exemplary indices of body mass index (BMI) and depths of an adipose layer associated with classifications for obesity and/or levels of health.

FIG. 2C depicts exemplary abdominal cross-sections obtained from human patients.

FIGS. 2D and 2E illustrate exemplary radio frequency (RF) testing performed by the Assignee hereof utilizing simulated human tissue and the implantable sensor apparatus described herein.

FIGS. 2F-2H illustrate results of the exemplary radio frequency (RF) testing of FIGS. 2D and 2E, including at different angles and distances relative to the simulated test subject.

FIGS. 2I and 2J are a pictorial illustration of direct and multipath propagation paths and temporal shifts for radio frequency energy from the exemplary sensor apparatus within a structure such as a building or apartment.

FIGS. 2K-1 and 2K-2 illustrate exemplary prior art radio frequency bands used with various short- and intermediate-range technologies (including BLE advertisement bands).

FIG. 2L is a table illustrating exemplary electrical power (battery) utilization due to various BLE advertisement schemes and operating modes for the exemplary sensor apparatus of FIGS. 1A-1E.

FIG. 2M is a graphical depiction of the various devices (implanted, transcutaneous and/or external) with which the exemplary sensor apparatus may wirelessly communicate data.

FIG. 2N is a graphical illustration of communication between the exemplary sensor apparatus and a parent platform, and a receiver platform (including parent-receiver communication for synchronization).

FIG. 3 is a logical block diagram illustrating one embodiment of a system architecture for, inter alia, monitoring blood analyte levels within a user, according to the present disclosure.

FIG. 3A is a logical block diagram illustrating another embodiment of a system architecture for, inter alia, monitoring blood analyte levels within a user, according to the present disclosure.

FIG. 3B is a logical block diagram illustrating yet another embodiment of a system architecture for, inter alia, monitoring blood analyte levels within a user, according to the present disclosure.

FIG. 3C is a logical block diagram illustrating a further embodiment of a system architecture for, inter alia, monitoring blood analyte levels within a user, according to the present disclosure.

FIG. 3D is a logical block diagram illustrating yet another embodiment of a system architecture for, inter alia, monitoring blood analyte levels within a user, according to the present disclosure.

FIG. 4A is a logical block diagram illustrating an exemplary implantable sensor apparatus and local receiver apparatus according to one embodiment of the disclosure.

FIG. 4B is a functional block diagram illustrating an exemplary embodiment of the local receiver apparatus.

FIG. 4C is a functional block diagram illustrating an exemplary embodiment of an integrated receiver and medicant delivery device.

FIG. 4D is a functional block diagram illustrating an exemplary embodiment of an external receiver apparatus communicative with an implanted medicant delivery and sensing device.

FIGS. 5A-5D are logical flow diagrams illustrating exemplary embodiments of methods of operating the analyte sensing system(s) described in the present disclosure.

FIGS. 6A and 6B are graphical illustrations of exemplary user interfaces including “blinded” graphical presentations.

All Figures © Copyright 2018-2019 GlySens Incorporated. All rights reserved.

### DETAILED DESCRIPTION

Reference is now made to the drawings, wherein like numerals refer to like parts throughout.

### Overview

One aspect of the present disclosure leverages Assignee's recognition that many disabilities of prior art “receiver” approaches for blood analyte monitoring by users (including the user being effectively tethered to their analyte monitoring system receiver, as well as unnecessary drain on power resources of the implanted sensor) can be effectively mitigated or even eliminated via specially configured apparatus and methods, such as those explicitly disclosed herein. Specifically, in one exemplary configuration, a sensor having low energy consumption features which enable long term implantation and improved accuracy of calculated data related to physiological parameters is disclosed. Further, such sensors are in some variants configured for computation and transmission of blood analyte data to one or more communicative devices, such as medicant delivery devices, calibration devices, receiver apparatus having a graphical user interface (GUI), and/or other electronic devices.

Further, the sensor in some implementations advantageously requires only opportunistic communication with an external device (e.g., a minimal profile and functionality receiving device which the user can discretely carry or wear continuously, a user's mobile device enabled with blood analyte receiver functionality, and/or a dedicated receiver apparatus, etc.). In other implementations, the sensor is additionally configured to operate in a training mode after implantation, and to generate and utilize a user-specific operational model for calculation of blood analyte data. Further, the processor of the sensor can store and apply one or more other error correction models or algorithms for correction of blood analyte data (e.g., random noise filter, temporal mismatch error correction algorithm, etc.). Yet further, the sensor may generate and/or store and utilize one or more pump error correction models for calculation of medicant dosage data which can be transmitted to a communicative computerized pump apparatus (e.g., an implanted pump, a non-implanted pump, etc.).

In one implementation, the aforementioned sensor is battery operated and is configured for ultra-low power consumption; power conservation is accomplished in one configuration through use of one or more of: (i) the foregoing opportunistic (i.e., non-continuous) communication with a receiver apparatus; (ii) use of Bluetooth Low Energy (BLE) for communication with external devices and/or other implanted devices, including enhanced receiver sensitivity (and hence reduced transmission power requirements); and/or (iii) reduced power modes for storage of the sensor prior to implantation.

Special construction features and technologies used in the sensor also further enhance RF signal transmission, reduce power consumption, and facilitate easy implantation and subsequent explant, as well enable efficient methods of manufacturing, testing, and reclamation.

Moreover, exemplary embodiments of the present disclosure advantageously enable user-facing smart-device advantages; for instance, the exemplary implanted sensor autonomously generates “final” blood glucose values that can be received by a plurality of independent and/or interconnected devices, thereby providing several user advantages including inter alia: (i) operational flexibility (e.g., not having to have a particular one of the user's plurality of mobile devices with them at all times), (ii) simplicity and consistency of data display on those various devices (i.e., each device can immediately display a simple value and/or graphical element(s) consistent with the others such that the user does not have to learn/remember different user interfaces), (iii) data integration with other health or related applications correlating, e.g., exercise and diet, with blood glucose levels, and (iv) enablement of different display paradigms and/or devices for different use models and user preferences (e.g. swim with a smart watch, at work with the user's smart phone, etc.). Yet further, the utilization of standard (e.g., BLE) wireless communication protocols, plus signal processing and blood glucose value generation in-vivo, liberates the user from proprietary receiver hardware.

Yet further, the implanted sensor can be controlled and updated via any of the external devices (with security provisions applied), including software/firmware updates over time (e.g., enhancements to extant onboard signal processing algorithms, more efficient code/firmware, better or upgraded BLE algorithms such as for wireless interface coexistence or interference mitigation in the ISM or other frequency bands of interest), thereby potentially obviating the need for another surgical procedure (i.e., explant and replacement of the sensor).

### Detailed Description of Exemplary Embodiments

Exemplary embodiments of the present disclosure are now described in detail. While these embodiments are primarily discussed in the context of a fully implantable glucose sensor, such as those exemplary embodiments described herein, and/or those set forth in U.S. Patent Application Publication No. 2013/0197332 filed Jul. 26, 2012 entitled “Tissue Implantable Sensor With Hermetically Sealed Housing”; U.S. Pat. No. 7,894,870 to Lucisano et al. issued Feb. 22, 2011 and entitled “Hermetic Implantable Sensor”; U.S. Patent Application Publication No. 2011/0137142 to Lucisano et al. published Jun. 9, 2011 and entitled “Hermetic Implantable Sensor”; U.S. Pat. No. 8,763,245 to Lucisano et al. issued Jul. 1, 2014 and entitled “Hermetic Feedthrough Assembly for Ceramic Body”; U.S. Patent Application Publication No. 2014/0309510 to Lucisano et al. published Oct. 16, 2014 and entitled “Hermetic Feedthrough Assembly for Ceramic Body”; U.S. Pat. No. 7,248,912 to Gough et al. issued Jul. 24, 2007 and entitled “Tissue Implantable Sensors for Measurement of Blood Solutes”; and U.S. Pat. No. 7,871,456 to Gough et al. issued Jan. 18, 2011 and entitled “Membranes with Controlled Permeability to Polar and Apolar Molecules in Solution and Methods of Making Same”; U.S. Patent Application Publication No. 2013/0197332 to Lucisano et al. published Aug. 1, 2013 and entitled “Tissue Implantable Sensor with Hermetically Sealed Housing”; and PCT Patent Application Publication No. 2013/016573 to Lucisano et al. published Jan. 31, 2013 and entitled “Tissue Implantable Sensor with Hermetically Sealed Housing,” each of the foregoing incorporated herein by reference in its entirety, as well as those of U.S. patent application Ser. Nos. 13/559,475; 14/982,346; 15/170,571; 15/197,104; 15/359,406; 15/368,436; 15/472,091; 15/645,913; 15/853,574; and 16/233,536, previously incorporated herein, it will be recognized by those of ordinary skill that the present disclosure is not so limited. In fact, the various aspects of the disclosure are useful with, inter alia, other types of implantable sensors, implantable pumps, and/or other electronic devices.

Further, while the following embodiments describe specific implementations of e.g., biocompatible oxygen-based multi-sensor element devices for measurement of glucose having specific configurations, protocols, locations, and orientations for implantation (e.g., sensor implantation proximate the waistline on a human abdomen with the sensor array disposed proximate to fascial tissue; see e.g., U.S. patent application Ser. No. 14/982,346, entitled “Implantable Sensor Apparatus and Methods” and filed Dec. 29, 2015, previously incorporated herein), those of ordinary skill in the related arts will readily appreciate that such descriptions are purely illustrative, and in fact the methods and apparatus described herein can be used consistent with, and without limitation: (i) in living beings other than humans; (ii) other types or configurations of sensors (e.g., other types, enzymes, and/or theories of operation of glucose sensors, sensors other than glucose sensors, such as e.g., sensors for other analytes such as urea, lactate); (iii) other implantation locations and/or techniques (including without limitation transcutaneous or non-implanted devices as applicable); and/or (iv) other devices (e.g., other sensor apparatus, medicant delivery devices, non-sensor devices, and non-substance delivery devices).

Moreover, while certain aspects of exemplary embodiments of the apparatus and methods of the disclosure are described with respect to Bluetooth® personal area networking technology (including e.g., BLE or Bluetooth Low Energy), it will be appreciated that use of the Bluetooth protocols and systems is merely exemplary and not a requirement for practicing such aspects of the disclosure. For example, another air interface technology (and associated protocols), whether within the same or different frequency band(s) as Bluetooth (e.g., 2.4 GHz ISM or other), may be used consistent with the disclosure, provided that the desired functionalities including sufficient signal permeation through biological tissue, are maintained.

As used herein, the term “analyte” refers without limitation to a substance or chemical species that is of interest in an analytical procedure. In general, the analyte itself may or may not be directly measurable, in cases where it is not, a measurement of the analyte (e.g., glucose) can be derived through measurement of chemical constituents, components, or reaction byproducts associated with the analyte (e.g., hydrogen peroxide, oxygen, free electrons, etc.).

As used herein, the terms “delivery device” and “medicant delivery device” refer to a device configured for delivery of solutes, including without limitation one or more mechanical or electro-mechanical pumps, such as partially implanted or fully implanted pumps, as well as other delivery modes such as diffusion (e.g., through a membrane or other barrier), or even dissolution of solids. Exemplary partially implantable pumps include transcutaneous pumps which include an implantable portion (e.g., a cannula, a needle, etc.) coupled to a non-implantable portion (e.g., a housing, a reservoir, a pump actuator, etc.). Exemplary fully implantable pumps include subcutaneous pumps, which are implanted beneath the skin of a user and are in data communication with an external controlling (e.g., processing) apparatus.

As used herein, the terms “detector” and “sensor” refer without limitation to a device having one or more elements (e.g., detector element, sensor element, sensing elements, etc.) that generate, or can be made to generate, a signal indicative of a measured parameter, such as the concentration of an analyte (e.g., glucose) or its associated chemical constituents and/or byproducts (e.g., hydrogen peroxide, oxygen, free electrons, etc.). Such a device may be based on electrochemical, electrical, optical, mechanical, thermal, or other principles as generally known in the art. Such a device may consist of one or more components, including for example, one, two, three, or four electrodes, and may further incorporate immobilized enzymes or other biological or physical components, such as membranes, to provide or enhance sensitivity or specificity for the analyte.

As used herein, the terms “orient,” “orientation,” and “position” refer, without limitation, to any spatial disposition of a device and/or any of its components relative to another object or being, and in no way connote an absolute frame of reference.

As used herein, the terms “top,” “bottom,” “side,” “up,” “down,” and the like merely connote, without limitation, a relative position or geometry of one component to another, and in no way connote an absolute frame of reference or any required orientation. For example, a “top” portion of a component may actually reside below a “bottom” portion when the component is mounted to another device (e.g., host sensor).

As used herein the term “parent platform” refers without limitation to any device, group of devices, and/or processes with which a client or peer device (including for example the various embodiments of local receiver described here) may logically and/or physically communicate to transfer or exchange data. Examples of parent platforms can include, without limitation, smartphones, tablet computers, laptops, smart watches, personal computers/desktops, servers (local or remote), gateways, dedicated or proprietary analyte receiver devices, medical diagnostic equipment, and even other local receivers acting in a peer-to-peer or dualistic (e.g., master/slave) modality.

As used herein, the term “application” (or “app”) refers generally and without limitation to a unit of executable software that implements a certain functionality or theme. The themes of applications vary broadly across any number of disciplines and functions (such as on-demand content management, e-commerce transactions, brokerage transactions, home entertainment, calculator etc.), and one application may have more than one theme. The unit of executable software generally runs in a predetermined environment; for example, the Java® environment.

As used herein, the term “computer program” or “software” is meant to include any sequence or human or machine cognizable steps which perform a function. Such program may be rendered in virtually any programming language or environment including, for example, C/C++, Fortran, COBOL, PASCAL, assembly language, markup languages (e.g., HTML, SGML, XML, VoXML), and the like, as well as object-oriented environments such as the Common Object Request Broker Architecture (CORBA), Java® (including J2ME, Java Beans, etc.) and the like.

As used herein, the terms “Internet” and “internet” are used interchangeably to refer to inter-networks including, without limitation, the Internet. Other common examples include but are not limited to: a network of external servers, “cloud” entities (such as memory or storage not local to a device, storage generally accessible at any time via a network connection, or cloud-based or distributed processing or other services), service nodes, access points, controller devices, client devices, etc.

As used herein, the term “memory” includes any type of integrated circuit or other storage device adapted for storing digital data including, without limitation, ROM, PROM, EEPROM, DRAM, SDRAM, DDR/2 SDRAM, EDO/FPMS, RLDRAM, SRAM, “flash” memory (e.g., NAND/NOR), 3D memory, and PSRAM.

As used herein, the terms “microprocessor” and “processor” or “digital processor” are meant generally to include all types of digital processing devices including, without limitation, digital signal processors (DSPs), reduced instruction set computers (RISC), general-purpose (CISC) processors, microprocessors, gate arrays (e.g., FPGAs), PLDs, state machines, reconfigurable computer fabrics (RCFs), array processors, secure microprocessors, and application-specific integrated circuits (ASICs). Such digital processors may be contained on a single unitary integrated circuit (IC) die, or distributed across multiple components.

As used herein, the term “network” refers generally to any type of telecommunications or data network including, without limitation, hybrid fiber coax (HFC) networks, satellite networks, telco or cellular networks, and data networks (including MANs, WANs, LANs, WLANs, internets, and intranets), cellular networks, as well as so-called “mesh” networks and “IoTs” (Internet(s) of Things). Such networks or portions thereof may utilize any one or more different topologies (e.g., ring, bus, star, loop, etc.), transmission media (e.g., wired/RF cable, RF wireless, millimeter wave, optical, etc.) and/or communications or networking protocols.

As used herein, the term “interface” refers to any signal or data interface with a component or network including, without limitation, those of the FireWire (e.g., FW400, FW800, etc.), USB (e.g., USB 2.0, 3.0. OTG), Ethernet (e.g., 10/100, 10/100/1000 (Gigabit Ethernet), 10-Gig-E, etc.), MoCA, LTE/LTE-A, 5G NR, Wi-Fi (802.11), WiMAX (802.16), Z-wave, PAN (e.g., 802.15)/Zigbee, 5G NR (3GPP), CBRS (Citizens Broadband Radio Service), Bluetooth, Bluetooth Low Energy (BLE) or power line carrier (PLC) families.

As used herein, the term “storage” refers to without limitation computer hard drives, memory, RAID devices or arrays, optical media (e.g., CD-ROMs, Laserdiscs, Blu-Ray, etc.), solid state devices (SSDs), flash drives, cloud-hosted storage, or network attached storage (NAS), or any other devices or media capable of storing data or other information.

As used herein, the term “Wi-Fi” refers to, without limitation and as applicable, any of the variants of IEEE-Std. 802.11 or related standards including 802.11 a/b/g/n/s/v/ac/ax or 802.11-2012/2013, as well as Wi-Fi Direct (including inter alia, the “Wi-Fi Peer-to-Peer (P2P) Specification,” incorporated herein by reference in its entirety),and Wi-Fi Aware.

As used herein, the term “wireless” means any wireless signal, data, communication, or other interface including without limitation Wi-Fi, Bluetooth (including BLE or “Bluetooth Smart”), NFC, 3G (3GPP/3GPP2), HSDPA/HSUPA, TDMA, CDMA (e.g., IS-95A, WCDMA, etc.), FHSS, DSSS, GSM, PAN/802.15, WiMAX (802.16), 802.20, Zigbee®, Z-wave, narrowband/FDMA, OFDM, PCS/DCS, LTE/LTE-A/LTE-U/LTE-LAA, CBRS (Citizens Broadband Radio Service), 5G-NR (3GPP), analog cellular, CDPD, satellite systems, millimeter wave or microwave systems, acoustic, and infrared (i.e., IrDA).

### Exemplary Implantable Sensor Housing and Sensing Region

Referring now to FIGS. 1A-1T, one exemplary embodiment of a sensor apparatus useful with various aspects of the present disclosure is shown and described.

As shown in FIGS. 1A-1J, the exemplary sensor apparatus 100 comprises a somewhat planar (and somewhat ovaloid) housing structure 102 having rounded opposing ends and substantially smooth and/or curved surfaces. A sensing region 104 is disposed on one side of the housing structure 102 (i.e., on a top face 102a, which opposes a bottom face 102b). The exemplary substantially planar shape of the housing 102 provides mechanical stability for the sensor apparatus 100 after implantation, thereby helping to preserve the orientation of the apparatus 100, mitigating any tissue response induced by movement of the apparatus while implanted, as well as increasing comfort of the host/user. Moreover, the rounded ends advantageously facilitate surgical insertion through one or more incisions formed within the subject as referenced elsewhere herein, and further give the apparatus 100 a less detectable and more comfortable profile after implantation due to, inter alia, the absence of sharp edges or corners. Further, the rounded opposing ends and substantially smooth and/or curved surfaces of the housing (as well as substantially eliminated seams between the housing components after assembly) aid in mitigation and/or limitation of foreign body response (FBR) after implantation of the sensor, thereby enabling long-term implantation and increasing accuracy of the sensor. Notwithstanding, the present disclosure contemplates sensor apparatus of shapes and/or sizes other than that of the exemplary apparatus 100.

The housing 102 of the illustrated embodiment includes two separable portions, a main body 106 and a cap 108. The main body 106 is comprised of a biocompatible metallic material, such as titanium or aluminum, which provides structural rigidity of the sensor housing and protects internal components of the sensor (as depicted in e.g., FIGS. 1F-1H). The structure of the main body 106 having an opening disposed therein (corresponding a location of the sensing region 104) is additionally depicted in FIGS. 1K-1L. The cap 108 is comprised of a signal transmissive material (i.e., one which has at least some permeability to electromagnetic radiation such as radio frequency signals in the desired band(s) of interest), such as ceramic or a polymer, which permits an antennae 110 (shown in and described in greater detail with reference to FIGS. 1O and 1T) to transmit and receive signals. The cap is joined to the main body at a sealed seam 116 (e.g., a welded seam). Advantageously, the use of a single seam (and welding thereof) helps maintain the fluid-tight (and air-tight) integrity of the apparatus after assembly and implantation.

As can be seen in FIGS. 1A-1E and 1M-1N, the cap 108 includes a grasping feature 112 and a plurality (two in this instance) of through-holes or anchor apparatus 114 disposed within an area of the grasping feature. In the depicted embodiment, the grasping feature comprises a curved and circumferential depression with the cap, which is utilized for manipulation of the sensor apparatus during manufacturing and/or implantation and can be “grasped” by hand, or with an appropriate tool (e.g., forceps). Anchor apparatus 114 provide the surgeon with the opportunity to anchor the apparatus to the anatomy of the living subject (via receipt of sutures (dissolvable or otherwise), tissue ingrowth structures, and/or the like therein), so as to frustrate translation and/or rotation of the sensor apparatus 100 within the subject immediately after implantation but before any tissue response (e.g., FBR) of the subject has a chance to immobilize (such as via interlock with the sensing region of the apparatus). See e.g., U.S. patent application Ser. Nos. 14/982,346 and 15/197,104, for additional details, considerations, and configurations regarding the aforementioned anchor apparatus and sensor implantation.

In alternate embodiments, the grasping feature and/or the anchoring apparatus may have different configurations. For example, the grasping feature may have ridged configuration or a textured surface to increase grip, and/or the anchoring apparatus may be raised above the surface of the end cap as e.g., eyelets (and not penetrate through a portion of its thickness as in the embodiment of FIG. 1D). In other examples, the grasping feature and/or the anchoring apparatus may be eliminated from the cap, giving the cap a substantially smooth surface. Further, anchoring features may additionally or alternatively be included in a different region of the housing, such as within or projecting outwardly from the main body. Furthermore, the anchoring features may have a gel-like substance disposed therein (e.g., a silicone or silicone-based compound) in order to discourage tissue growth into or within the anchoring features, thereby easing a subsequent explant of the sensor and limiting damage to surrounding tissues during the explant procedure.

As can be seen in e.g., FIGS. 1D and 1G, the sensor apparatus further includes a plurality of individual sensor elements 118 with their active surfaces disposed substantially within the sensing region 104 on the top face 102a of the apparatus housing. In the exemplary embodiment (e.g., an oxygen-based glucose sensor), the five (5) sensing elements 106 are disposed in groups on the sensor face, one element of each group being an active or “primary” sensor with enzyme matrix, and the others being reference or “secondary” (oxygen) sensors associated with the proximate primary sensor (which are unassociated with any enzyme matrix). Exemplary implementations of the sensing elements and their supporting circuitry and components are described in, inter alia, U.S. Pat. No. 7,248,912, U.S. patent application Ser. Nos. 15/170,571; 15/359,406; and 16/233,536, each previously incorporated herein.

It will be appreciated that the type and operation of the sensor apparatus may vary; i.e., other types of sensor elements/sensor apparatus, configurations, and signal processing techniques thereof may be used consistent with the various aspects of the present disclosure, including, for example, signal processing techniques based on various combinations of signals from individual elements in the otherwise spatially-defined sensing elements pairs. Moreover, other exemplary embodiments of the sensor apparatus described herein may include any of: (i) multiple detector elements which can have respective “staggered” ranges/rates of detection operating in parallel, and/or (ii) multiple detector elements, optionally having respective “staggered” ranges/rates of detection, that are selectively switched on/off in response to, e.g., the analyte concentration reaching a prescribed upper or lower threshold, or a certain sensor group or type being optimal under specific conditions, such as those described in the foregoing U.S. patent application Ser. No. 15/170,571, previously incorporated herein. The present disclosure further contemplates that such thresholds or bounds, and/or sensor groups: (i) can be selected independent of one another; and/or (ii) can be selected dynamically while the apparatus 100 is implanted. For example, in one scenario, operational detector elements are continuously or periodically monitored to confirm accuracy, and/or detect any degradation of performance (e.g., due to equipment degradation, progressive FBR affecting that detector element, etc.). If degradation is detected, e.g., affecting say a lower limit of analyte concentration that can be detected, a particular detector element can be “turned off” or have its signals removed from data calculations, such that handoff to another element capable of more accurately monitoring concentrations in that range or under those specific physiological conditions occurs. Note that these thresholds or bounds are to be distinguished from those associated with the user interface (UI) described subsequently herein, the latter being independent of the data source/capability/configuration associated with the sensor detector elements.

Additionally or alternatively, embodiments of the presently described sensor apparatus can include heterogeneous sensor elements of first and second types, such as e.g., oxygen-based sensor elements and hydrogen peroxide-based sensor elements, each configured for detection of blood glucose. In one such implementation, the implantable sensor apparatus includes both oxygen-based sensor elements and hydrogen peroxide-based sensor elements, one of the element types acting in a confirmatory or calibration capacity to in effect “second check” and adjust (as necessary) the other sensor elements, thereby ostensibly extending the interval between other confirmatory processes utilized with the device (e.g., fingersticking), and/or the implant-to-explant interval, hence improving user experience with the device and quality of life.

In one exemplary configuration, the first and second analyte detector element types are used in parallel, and one detector type acts as a reference for the other detector type (e.g., the hydrogen peroxide-based glucose detector element is a reference for the oxygen-based glucose detector element, the oxygen-based glucose detector element is a reference for the hydrogen peroxide-based glucose detector element, etc.). In another example, the first and second analyte detector element types are used in parallel and measurements or readings from both detector element types are used to generate or derive a composite measurement (e.g., via a weighted average). In yet another example, the first and second analyte detector types can be alternately and/or selectively employed depending on specific implantation, use, and/or physiological conditions.

In one variant, the various heterogeneous detector elements (e.g., detector elements of the first glucose detector type and the second glucose detector type, and/or detector elements of various configurations for either sensor type according to specified ranges of sensitivity and/or rates of detection) can be selectively switched on/off (even while the sensor apparatus is in vivo), so as to, e.g., accommodate “on the fly” changes to blood glucose concentration or other physiological changes occurring within the host, or to maintain efficacy of the detector elements within a known or desirable range of accuracy or sensitivity. One type of detector can also be prioritized over another, or swapped out, such as e.g., where the performance of one detector type has eroded over time (due to e.g., FBR associated with that particular detector), or loss of some other desirable attribute or performance aspect. Specific examples of the foregoing heterogeneous detector element sensors are shown and described in the U.S. patent application Ser. No. 15/359,406, previously incorporated herein. It will be appreciated that an on-board processor of the sensor and its associated logic can be configured to perform the foregoing selective use (e.g., turning on/off) of sensor elements.

Returning to FIGS. 1D and 1G, in addition to being configured to have (either homogenous or heterogeneous) sensing elements disposed therein, the sensing region 104 may be configured to facilitate some degree of “interlock” of the surrounding tissue (and any subsequent tissue response generated by the host) so as to ensure direct and sustained contact between the sensing region 104 and the blood vessels of the surrounding tissue during the entire term of implantation (as well as advantageously maintaining contact between the sensing region 104 and the same tissue; i.e., without significant relative motion between the two). See e.g., U.S. patent application Ser. No. 15/197,104 filed Jun. 29, 2016 and entitled “Bio-adaptable Implantable Sensor Apparatus and Methods,” previously incorporated herein, for additional details and considerations regarding utilization of the user's foreign body response (FBR) to at least partially generate interlock between the sensor face and the host tissues.

It will be appreciated that the relatively smaller dimensions of the sensor apparatus (as compared to many conventional implant dimensions)—on the order of 53 mm in length (dimension “a” on FIG. 1G) by 22 mm in width (dimension “b” on FIG. 1G) by 7 mm in height (dimension “c” on FIG. 1G)—may reduce the extent of injury (e.g., reduced size of incision, reduced tissue disturbance/removal, etc.) and/or the surface area available for blood/tissue and sensor material (i.e., non-active portions of the sensor) interaction, which may in turn reduce intensity and duration of the host wound healing response, and increase longevity of the implant due to greater signal stability over time. It is also envisaged that as circuit integration is increased, and component sizes (e.g., Lithium or other batteries) decrease, and further improvements are made, the sensor may increasingly be appreciably miniaturized, thereby further leveraging this factor.

### Exemplary Implantable Sensor Internal Components

As discussed supra, the housing is configured to enclose, protect and provide structural support for various internal components of the sensor. The internal components are disposed on a circuit board 120, depicted in FIGS. 1I-1J and 1N-1P. As used herein, the terms “board” and “circuit board” are intended to include any structure which provides such functionality, including without limitation (i) assemblies of two or more boards or components, and/or (ii) other substrate-like components, whether rigid, flexible, or other (e.g., “flex” boards).

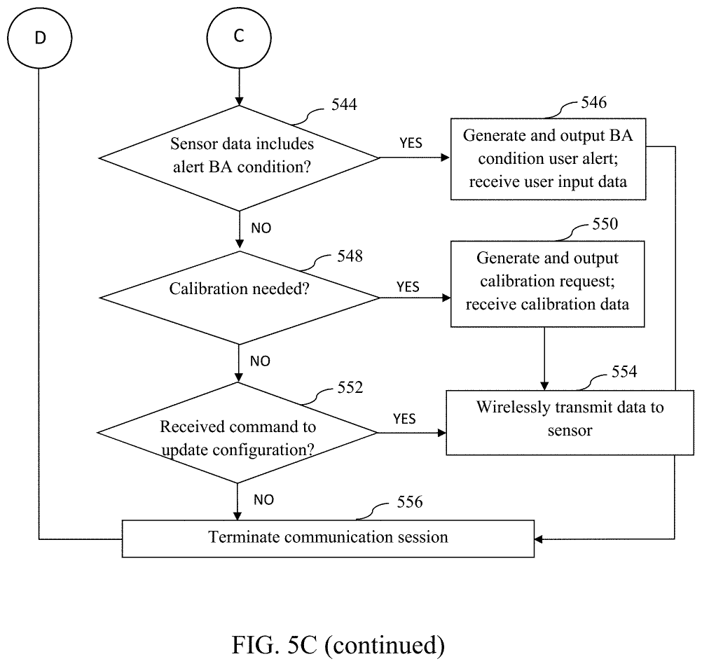

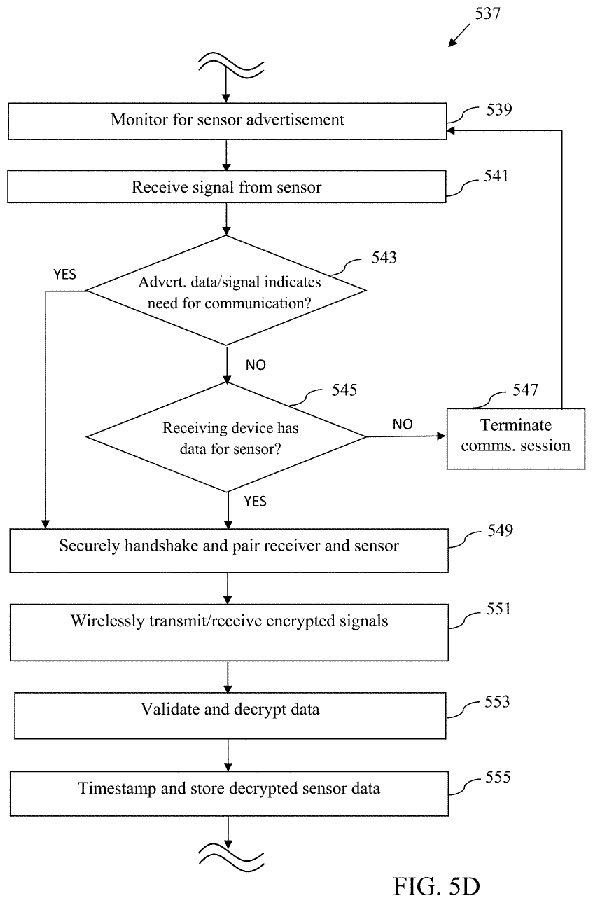

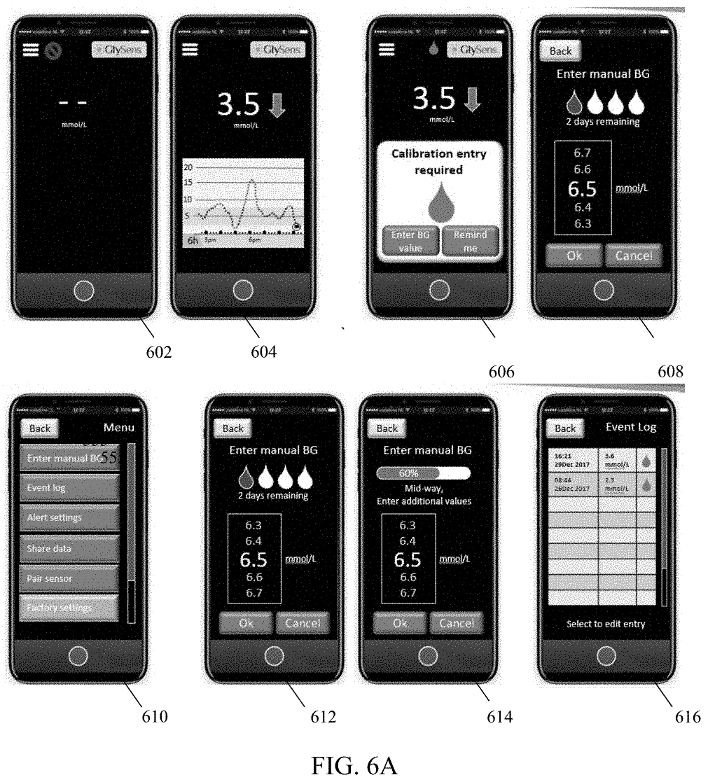

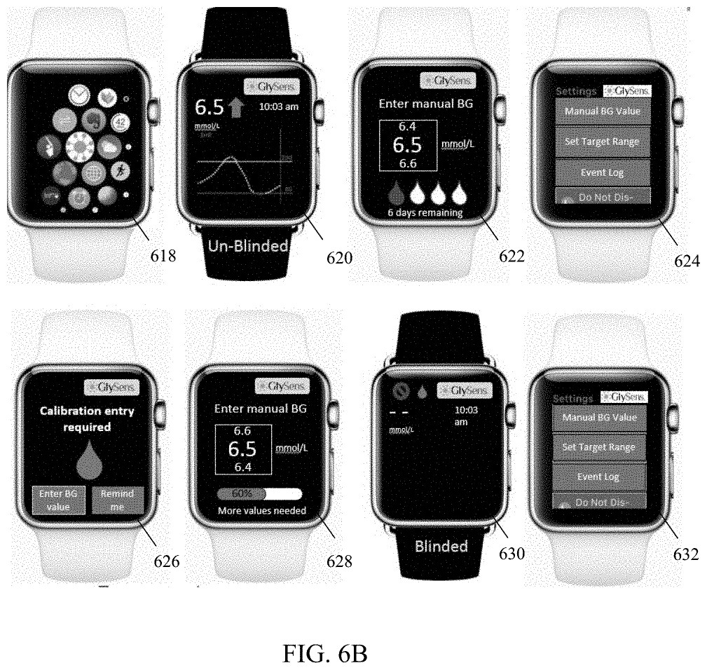

As can be seen in the cross-sectional views of FIG. 11 (taken along the line I-I shown in FIG. 1F) and FIG. 1J (taken along the line J-J shown in FIG. 1H), the circuit board 120 comprises a planar body 122 having a top surface 120a, proximate to the top face 102a of the sensor housing, and a bottom surface 120b, proximate to the bottom face 102b of the sensor housing.

The planar body 122 has an oval shape (when observed from a top plan view), substantially matching internal dimensions and shape of the housing 102. Abutment of a perimeter edge 124 of the planar body to an interior wall 126 of the housing enables a position the circuit board to be retained within an interior space 128 the housing. Additional support and retention of the circuit board (e.g., limitation of rotational movement within the interior space 128) is provided by a pair of flanges 130 disposed at opposing lateral sides of the circuit board, which are each mated with a shoulder 132 on an interior wall 134 of the (e.g., ceramic) cap 108. Further, the interior wall 134 of the ceramic cap includes a groove 136 disposed therein and configured to receive at least a portion of the perimeter edge 124 of the circuit board, thereby further constraining movement of the circuit board within the interior space of the housing (e.g., limitation of horizontal movement within the interior space 128).

FIGS. 1O and 1P respectively show internal components mounted on the top surface 120a and the bottom surface 120b of the circuit board. As can be seen in FIG. 1O, the top surface 120a includes a mounting region 138 having an opening 140 disposed therein for mating of a sensor element body (e.g., sensor disc 156 shown in FIGS. 1R and 1S) thereto. The sensor elements disposed within the sensor element body are configured to be electrically coupled with contacts 142 disposed on the bottom surface 120b. The contacts 142 are configured to deliver sensor signals to a microprocessor and BLE unit 144, which is attached at the top surface 120a. The top surface (in addition to microprocessor and BLE unit 144) further includes the antenna 110, a potentiostat 146, a humidity sensor 148, and a thermistor 150, while the bottom surface 120b further includes an accelerometer 160 and a reed switch 162, each in signal and/or data communication with the microprocessor and wireless (e.g., BLE) unit 144.

In one implementation, the microprocessor and BLE unit 144 is an ultra-low power processor, such as e.g., a Nordic Semiconductor' nRF52840, supporting Bluetooth 5. The ultra-low power processor utilizes power and resource management to maximize application energy efficiency and battery life of the implanted sensor apparatus. For example, a power supply range between 1.7V and 5.5V can support primary and secondary cell battery technologies and direct USB supply without the need for regulators. Further, all peripheral components in data communication with the ultra-low power processor in the exemplary implementation include independent and automated clock and power management to ensure that they are each powered down when not required for task operation, thereby keeping power consumption to a minimum without the application having to implement complex power management schemes. Data transmitted to and from the ultra-low power processor is also optionally encrypted by via an on-board encryption system, such as e.g., an ARM CryptoCEll cryptographic system on chip and/or a full AES 128-bit encryption suite. It will be appreciated that one or more other BLE-capable microprocessors and/or ASICs may be additionally or alternatively used in the internal components of the sensor apparatus 100.

The aforementioned internal components of the sensor apparatus are each powered by a power source 152 (depicted in FIGS. 1Q, 1R and 1S), which is electrically coupled to the bottom surface 120b. Accordingly, the bottom surface 120b further includes contacts 154 and 155 for electrical coupling of the power source 152 to the circuit board. In one implementation, the power source comprises a coin cell battery 152a (depicted FIG. 1Q) coupled to contacts 154. In another implementation, the power source comprises a custom-shaped lithium battery 152b (depicted in FIGS. 1R and 1S) having a casing which is specifically configured to fit (and substantially fill) an upper portion 158 of the interior space of the housing so as to maximize a volume (and power capacity) of the power source, and is configured to couple to contact 155. It will be appreciated that the circuit board can be configured to mate with either power source type (either of battery 152a or 152b), or may be configured to mate with a single power source type.

As a brief aside, the aforementioned reed switch 162 may be utilized (in combination with other control features) to select/regulate various “power modes” of the assembled sensor, which enable power conservation prior to implantation and use of the sensor. In one embodiment, the reed switch (and/or other control features) are configured to enable four power modes of the sensor apparatus 100. A first power mode is configured for long-term storage of the sensor. In one implementation, the modes include: (i) a first power mode (i.e., a long-term storage mode) comprising the presence of a magnet (for maintaining a disconnected state of reeds within the switch), which enables “ultra-low” power consumption (e.g., 0.5 uA, utilizing 1.1% of the battery per year); (ii) a second power mode (i.e., a normal storage mode) comprising removal of the foregoing magnet, which enables “low” power consumption (e.g., 2 uA, utilizing 0.4% of the battery per month) and low power software utilization; (iii) a third power mode (i.e., a ready for use mode) where low power software utilization and high latency Bluetooth/BLE advertising are enabled at a higher power consumption (e.g., 4 uA, utilizing 0.8% of the battery per month); and a fourth mode (i.e., a run mode) where full power software utilization and normal Bluetooth/BLE advertising are enabled at a highest power consumption (e.g., 22 uA, utilizing on the order of 4.4% of the battery per month). The foregoing is merely exemplary and, in alternate embodiments, the sensor apparatus may be configured to operate in fewer or additional power modes, such as for example to accommodate other operational scenarios. In one implementation, the power modes may be programmed after implantation (e.g., such as via upload or “flashing” of an onboard program memory or firmware via the wireless interface).

Returning to FIG. 1P, the circuit board additionally includes a breakaway tab 164 and test point contact 166 for use during programming and/or testing of the assembled circuit board, which enable early identification and debugging of electrical signaling between the various components. After testing and programming, the breakaway tab 164 is configured to be removed from the circuit board along a score line 168, and the power source 152 can then be coupled at the corresponding electrical contacts (as depicted in FIG. 1Q in an exemplary implementation including the coin cell battery 152a).

### Sensor Apparatus Assembly

Turning now to FIGS. 1R and 1S, the exploded views demonstrate assembly of an exemplary implementation of the sensor apparatus 100 including the custom lithium battery 152b. In this implementation, the sensor disc has been pre-mounted and electrically coupled to the circuit board with e.g., a transfer adhesive and a silver epoxy. The circuit board is additionally wire bonded to the disk and the wire bonds are encapsulated with epoxy. In the exemplary views of FIGS. 1R and 1S, the breakaway tab has been previously removed (after testing and programming of the circuit board, as discussed supra).

Next, the custom battery 152b is mounted and electrically coupled to the circuit board at contact 155 disposed on the surface 120b. Specifically, the battery is soldered to the circuit board at the corresponding electrical contacts. A brief functional test of the connected battery may be performed prior to inserting the assembled circuit board into the main body portion 106 of the housing. In some examples, the microprocessor is functionally tested with an initial or lower complexity software program during assembly of the sensor apparatus.

Although not specifically shown, during insertion of the board, the sensor disc is positioned such that it is aligned with and extends through the opening in the housing 106 (corresponding to the sensing region 104). After insertion of placement of the circuit board, the cap 106 is placed on the end of the circuit board, thereby mating the flanges 130 of the circuit board to the shoulders 132 within the interior wall of the cap. The assembled sensor is sealed, such as via e.g., baking the completed sensor apparatus and laser welding around the sensor disc and at the sealable seam 116 between the main body 106 of the housing and the cap 108.

In one exemplary alternative implementation, the sensor board 120 is inserted into the housing 106 prior to insertion/attachment of the battery 152b. In such an implementation, the custom battery may have a larger configuration (i.e., a larger volume), thereby enabling e.g., a higher energy capacity of the battery and a longer implantation lifetime for the sensor apparatus.

Additional manufacturing steps carried out on the sealed sensor apparatus include, but are not limited to: leak testing, installation of membranes in the sensing region, programming (or updating and re-programming) and booting of the microprocessor and/or other computerized components, functional testing, calibration, sterilization, packaging, and storage.

### Exemplary Wireless Communication Interface

As discussed supra, the sensor apparatus 100 includes a wireless interface, comprising a data communication interface of the microprocessor and BLE unit 144 and antennae 110. As can be seen in FIG. 1T, in one implementation, the antenna 110 includes two partly linear and partly arcuate traces 170. The two linear/arcuate traces extend over one end of the top surface 120a of the circuit board and are configured to transmit and receive signals primarily through the ceramic end cap 108 of the sensor housing. In the exemplary embodiment, the 2.4 GHz band is used as the basis of the BLE interface (see FIGS. 2K-1 and 2K-2) due to its general ubiquity and compatibility with personal electronics such as extant smartphone and/or smartwatch interfaces, although other frequency bands and/or interfaces may be used (such as e.g., 802.15.4) provided that maintain sufficient functionality as described elsewhere herein. Further, the antennae circuit is optimized or impedance-matched for transmission efficiency when the sensor apparatus is disposed within tissues of the living entity (e.g., human tissues).

The antenna form factor of the illustrated embodiment also advantageously is disposed on the planar substrate (via printing or other such deposition techniques) and as such, maintains a planar form factor that is generally parallel to the plane of the end cap, and which approximates the radius of the end cap such that the arcuate portions generally track along the inner periphery of the curved end cap. As such, maximal trace length and antenna efficiency are maintained while obeying the very small form factor of the implantable sensor apparatus.

Advantageously, the Bluetooth Low Energy (BLE) protocols, such as Bluetooth 4.x or Bluetooth 5, enable low power communications with external devices (e.g., an external receiver/processor apparatus, a computerized calibration device, an external computerized medicant delivery device, etc.) and/or other implanted devices (e.g., an implanted receiver/processor apparatus, an implanted medicant delivery device, etc.) within a relatively large communications range, and even as the signals move through tissues (e.g., muscle, adipose, and/or cutaneous tissue layers) of the host. Specifically, the wireless interface is configured to periodically transmit beacon data therefrom (e.g., every 10 seconds), and to pair with another device for wireless data communication through the user's tissues if certain conditions are met (discussed in detail infra with reference to FIGS. 5A-5D). Thus, transmission of signals through the user's tissues is necessary for data communication with the implanted sensor apparatus.

FIGS. 2A and 2B respectively show exemplary indices 200 and 202 of body mass index (BMI) and depths of an adipose layer associated with classifications for obesity and/or levels of health. FIG. 2C depicts exemplary abdominal cross sections 204 from patients, showing a cutaneous layer 206, an adipose layer 208, and a deep fascial layer 210 enclosing an abdominal core 212 (comprising muscle, visceral fat, and internal organs), and regions of interest for subcutaneous fat areas (top figure) 213 and visceral fat areas (bottom FIG. 214. It will be appreciated that a typical user/patient typically has a 1-2 mm cutaneous (skin) layer, a 1-3 mm adipose (fat) layer, while the core has a diameter of approximately 30 cm. However, certain diseases which are managed via an implantable sensor, such as e.g., diabetes, often contemporaneously present and/or are associated with obesity.

In one exemplary embodiment, as disclosed in U.S. patent application Ser. No. 14/982,346 (previously incorporated herein), the implantable sensor is configured to be implanted deep within the being's torso subcutaneous tissue proximate the extant abdominal muscle fascia, and oriented so that the sensing region faces away from the user's skin surface (i.e., the plane of the sensor is substantially parallel to the fascia and the epidermis/dermis, with the sensing region facing inward toward the musculature under the fascia). Therefore, the ability of the implanted sensor transmit and receive data through even abnormally thick adipose layers (e.g., 30-60 mm adipose tissue layers) is desirable.

In one exemplary scenario of data communication of an implanted sensor and an external receiver apparatus (shown in FIGS. 2D and 2E), the sensor 100 is disposed within a cavity or pocket formed within a living being 215 (e.g., implanted at the frontal portion of a human, more specifically the abdomen, proximate the waistline—simulated human shown), while a receiver apparatus 700 (e.g., a personal mobile device) is disposed external of the living being at an approximate 2 m distance from the living being. In the depicted scenario, the implanted sensor is substantially implanted below approximately 3 mm of cutaneous tissue, 7-60 mm of adipose tissue, and 0-3 cm of muscle tissue. As can be seen in the table 216 shown in FIG. 2F, in one exemplary implementation, total reception-free space for signal reception by the external receiver apparatus at a 2 m distance is 90% for a sensor implanted below 7 mm of adipose tissue, 88% for a sensor implanted below 30 mm of adipose tissue, and 83% for a sensor implanted below 60 mm of adipose tissue. Further, corresponding radiation efficiencies of the sensors are −25.6 dBi, −28.6 dBi, and −31.3 dBi, respectively.

Models of the foregoing data are shown in FIGS. 2G and 2H. Specifically, one exemplary signal reception data model 218 is depicted in FIG. 2G, and an exemplary three-dimensional radiation efficiency model 220 is depicted in FIG. 2H. As can be seen in each of the models 218, 220 reception and radiation of signals have highest efficiency in a space in front of and to lateral sides (e.g., 330-210 degrees) of a user who has a sensor implanted at a front their torso. As the signal reception and radiation are measured towards the rear of the user (e.g., 210-265 and 275-330 degrees), efficiencies decreases (except at an area directly behind the user, such as at e.g., 270 degrees).

Further, efficiency is inversely related to the depth of the adipose layer within which the sensor is implanted, and is thereby affected and limited by the depth the adipose layer through which signals are transmitted. Accordingly, as shown in the exemplary model 218, at a depth of 7 mm of fat, reception at a 6 m distance extends from about 310 to 230 degrees; at a depth of 30 mm of fat, reception at a 6 m distance extends from about 320 to 220 degrees; and at a depth of 60 mm of fat, reception at a 6 m distance extends from about 330 to 210 degrees.

The above data is noteworthy from at least the standpoint that using the exemplary implanted sensor apparatus 100 disclosed herein equipped with BLE transceiver(s) and implanted to a typical depth within the abdomen of a human, a high degree of RF communications signal strength is achievable at distances within which a normal user's personal electronics device (e.g., smartphone) would normally be maintained, such as in their pocket, purse, backpack, on a table nearby at dinner, or at night while sleeping.

It is further recognized by the inventors hereof that signal reception at the receiving apparatus can be improved in some operational scenarios via multipath propagation. Specifically, signals can be received by the antenna of the receiving apparatus along two or more pathways, which are caused by signals reflecting off of objects (e.g., walls, ceiling, obstructions, etc.). To some degree, such “objects” may even include internal structures of the host being (e.g., bones), such that multiple egress signal paths from the host may exist. As can be seen in FIG. 2I, multipath models 222, 224 demonstrate that reflection of signals off of a hypothetical surface (e.g., a ceiling) and an obstruction can be combined with directly transmitted signals to increase the received signal strength (i.e., via signal addition processing such as e.g., via MIMO or other spatial diversity techniques, or via constructive interference). Thus, as depicted in FIG. 2J, signal strength of an implanted sensor 100 is increased via use in an environment 226 which includes surfaces and objects (such as e.g., walls, tables, counters, etc.) such as a user's premises or office. Data obtained by the Assignee hereof indicates that use of the implanted sensor and receiver in the environment 226 can increase signal reception of the external receiver apparatus 700 up to 100% at a 22 ft. distance, even through one or more walls, and/or in various orientations of the receiver apparatus (e.g., laying flat, upright, etc.).

As can be seen in diagram 228 of FIGS. 2K-1 and 2K-2, BLE devices, such as the foregoing exemplary embodiments of the sensor 100, are detectable through a procedure based on broadcasting advertising packets using 3 separate channels (frequencies) 230, in order to reduce interference. The advertising device (e.g., the implanted sensor apparatus) sends packet data (e.g., beacon data) on at least one of these three channels, with a repetition period or advertising interval. For reducing the chance of multiple consecutive collisions, a random delay of up to 10 milliseconds is added to each advertising interval. A receiving device monitors the channel(s) for a duration called the scan window, which is periodically repeated every scan interval.

The sensor 100 may be additionally enabled for coded PHY operations, such as e.g., Long-Range Mode (LRM) operation. Such operation can improve sensitivity of the implanted sensor receiver and communications range, but may require a power trade-off (i.e., power consumption is increased in LRM). In some examples according to the present disclosure, the sensor may selectively implement LRM operation under specific conditions where no external receiver is in range and communication is required (i.e., a BA alert condition is present). The decision to implement this mode is in one instance driven by computerized logic executing on the sensor apparatus processor core; i.e., when no “handshake” or other protocol session is established between the sensor apparatus transceiver and an acceptable external receiver (e.g., one with the capability to receive and decode and utilize the blood analyte data/alert data) and an alert condition exists, the logic causes the sensor apparatus to enter the LRM mode for at least a period of time. A table 230 of exemplary BT advertisement intervals and power requirements (including LRM mode) is shown in FIG. 2L. As shown, the percentage of (sensor) battery power consumed when in LRM operation increases substantially as the advertisement interval decreases. As such, exemplary embodiments of the sensor of the present disclosure may also be equipped with logic to vary the advertisement interval in a prescribed manner (e.g., start with the lowest energy-consuming rate, and increase accordingly over time and number of advertisements transmitted until communication established). Moreover, such logic may also take into account the proximity of the battery (or batteries) to their EOL, such as may be determined by variation of a voltage profile over time during implantation, and/or by total Ah (Amp hours) consumed relative to a nominal battery profile or capacity (e.g., 428 mAh in FIG. 2L). Specifically, batteries may exhibit a non-linear curve of voltage decay over time, and may nominally provide the “nameplate” capacity in terms of mAh, and as such the frequency/mode of LRM operation may be adjusted based on proximity to the extrapolated EOL of the battery, such that precious remaining battery assets are not unnecessarily used, thereby potentially causing explant of the sensor 100 earlier than necessary.

The duration of implantation may also be monitored and utilized by such logic in evaluation of how rapidly the sensor is utilizing electrical power relative to its target implantation duration. For instance, in one implementation, if the sensor is intended to be implanted for at least two (2) years, and the battery capacity is sized accordingly, the battery should, under all reasonable operating conditions, maintain sufficient voltage/current profile until well after the actual explant of the sensor. However, if the aforementioned sensor logic detects over time (e.g., at certain prescribed points during the implantation period) that the rate of battery depletion is exceeding that consistent with the target implantation period, the logic may selectively “clamp” comparatively high power consumption activities such as high-frequency LRM advertisements so as to attempt to further extend battery life.

Additionally or alternatively, as described in the aforementioned documents incorporated herein, the wireless data communication interface (i.e., transmitter/transceiver of the sensor apparatus) may be configured to transmit modulated radio frequency (RF) signals to a partially or fully implanted or in vivo receiving device, such as an implanted pump or other medication or substance delivery system (e.g., an insulin pump or other medicant dispensing apparatus), embedded “logging” device, or other. It is also appreciated that other forms of wireless communication may be used for such applications, including for example inductive (electromagnetic induction) based systems, or even those based on capacitance or electric fields, or even optical (e.g., infrared) systems where a sufficiently clear path of transmission and reception exists, such as two devices in immediately adjacent disposition, or even ultrasonic systems where the two devices are sufficiently close and connected by sound-conductive media such as body tissues or fluids, or a purposely implanted component. Further, it will be appreciated that a specific form of wireless communication can be selectively utilized (as determined by the sensor apparatus) with different devices and/or under differing conditions where one form is preferable over others.

Exemplary Bluetooth-enabled devices configured for data communication with the sensor apparatus 100 are depicted in FIG. 2M. Specifically, the sensor 100 is configured for data communication via its BLE interface with one or more of a local receiver 400 (e.g., a wearable receiver), a parent platform 700 (e.g., a personal consumer electronic device such as a user's mobile phone or tablet), a computerized apparatus 702 (e.g., a user's lab top or desk top computer), a dedicated receiver and processor apparatus 450, transcutaneous medicant pumps 1000 and 1002 (e.g., a tethered medicant pump, a patch medicant pump, etc.), an implanted medicant pump receiver and processor apparatus 1020, an implanted medicant pump 1020a, and/or a computerized calibration device 840.

Moreover, the Bluetooth-enabled devices shown in FIG. 2M may be additionally configured for data communication with each other and/or external networks (e.g., network locations, servers, and storage locations) via one or more wireless communication protocols, such as Bluetooth, other personal area networks (PAN), a local area network (LAN), a wide area network radio (WAN), broadband cellular network, and/or radio frequency (RF) signals, or via wired communication. In one specific example depicted in FIG. 2N, the implanted sensor apparatus 100 requires (at least periodic) data communication with the parent platform 700 via BLE. Optionally, the implanted sensor apparatus 100 can communicate with a wearable local receiver apparatus 400. When the local receiver apparatus is utilized, the parent platform 700 and the local receiver apparatus 400 are configured to periodically synchronize (synch) their respective received sensor data when the parent platform and the local receiver are within communications range of or connectivity with each other (such communications which may be wireless or wireline-based). In one implementation, if new data is present on either device, the devices 700, 400 may automatically synchronize their received sensor data. In other implementations, the parent platform and the local receiver may synchronize data when commanded by a user, and/or at pre-determined intervals.

In one embodiment, the parent platform 700 is configured such that it maintains a comparatively large data storage capacity, and has cloud connectivity (e.g., via cellular LTE/LTE-A or 5G NR modem or WLAN or WMAN interface) such as for user data uploads for distribution to other user devices (e.g., a user's home PC), cloud data repositories or other storage/analytical entities, and/or downloads such as firmware updates, application software (“app”) updates, new calibration/analytical values, archived historical data, etc. In one variant, the user device app is configured to generate user-specific BG values, alerts (whether generated by the implanted sensor, or the user device), trend data, and other useful information (e.g., when “unblinded” or enabled by the user, such as when privacy/discretion is not required). Third-party databases (such as e.g., HealthKit) may also be enabled for data interchange via the user device app.

Likewise, an app operative to run on the receiver 400 may include functionality relating to, inter alia, BG value inputs by the user (such as for calibration), and may be used to provide the user (similarly when “unblinded” as with the parent platform app) with BG values, alerts, trends or other useful data. As such, the receiver app (and receiver 400) comprise a less fully featured function set than the parent platform, and can be useful for example when use or possession of the parent platform is not practicable (e.g., when engaging in sporting activities including watersports, when possession or use of a cellular or Wi-Fi-enabled smartphone is prohibited or it must be turned off, or simply when the user forgets it during their travels).

It is also recognized that all or portions of the extant Bluetooth “Health Care” profiles (i.e., GLP (Glucose Profile)—for blood glucose monitors, and CGMP (Continuous Glucose Monitor Profile)) may be used consistent with the present disclosure, such as via a sensor apparatus 100 configured to expose the CGM Service, with the sensor apparatus acting as a GATT Server, and an external (or internal) receiver 400, 700 acting as a Collector. See “Continuous Glucose Monitoring Profile Bluetooth Profile Specification” dated 2014-Nov.-18 Revision V1.0.0, incorporated herein by reference in its entirety.

It will also be appreciated that while many exemplary implementations of the sensor apparatus 100 are described herein with respect to Bluetooth-based technologies such as BLE, other comparable technologies may be substituted as applicable/desired, depending on the particular application, desired implantation longevity, desired communications range, and any other number of factors including the external body (housing) and end cap material selected. For instance, 3GPP NB-IoT based transceivers may be used, as may IEEE 802.15.4. At present, Bluetooth advantageously has a great deal of ubiquity in consumer electronic devices, thereby making it a good choice from that perspective. However, it is envisaged that other technologies may become more pervasive over time, including 5G NR-based IoT, and with comparable low electrical power consumption, can be used consistent with the disclosure. It is also appreciated that multi-mode or SDR (software defined radio) based solutions may be used as part of the sensor apparatus, such that a given sensor can, when implanted, communicate with external devices (or even other internal implanted devices such as medicant delivery systems) via two or more different air interfaces.

### Sensor System Architectures

As described elsewhere herein, exemplary embodiments of the present disclosure utilize communication with a fully implanted analyte sensor apparatus to, inter alia, monitor a blood analyte level of a user, and in certain embodiments, utilize blood analyte data provided by the implanted sensor to affect substance delivery, such as via medicant delivery devices (e.g., insulin pumps) and/or manual/semi-manual medicant delivery mechanisms (e.g., subcutaneous insulin injection via a syringe or a computerized insulin injection pen). Notably, data communication with the implanted sensor may be selective or opportunistic in nature (or may be continuous in nature, if desired) and utilizes a low power consumption data communication interface (e.g., PAN such as BLE) such that battery resources of the implanted sensor are preserved (thereby e.g., enabling even longer term implantation of the sensor). Further, processing of blood analyte data may be primarily implemented in computerized logic (software, firmware, or even hardware) that is resident within the implanted sensor apparatus itself (which may supplemented by or alternatively be resident in any number of different locations within the system, including: (i) “off-board” the sensor apparatus, such as in an external receiver apparatus (examples of which are described below); (ii) off-board, in a connected “cloud” entity; and/or combinations of the foregoing (e.g., in a distributed computing architecture); or (iii) off-board, in another implanted apparatus (e.g., an implanted medicant delivery device, a data logging device, etc.)). Accordingly, the following embodiments are merely examples of such types of architectures, and the various aspects of the present disclosure are in no way limited thereto.

As shown in FIG. 3, a first system architecture 300 is shown, wherein the implanted sensor apparatus 100 communicates directly with an external receiver or host platform 400 such as an appropriately equipped smartphone (e.g., one with sufficient software/app to receive, extract, and utilize data transmitted from the implanted sensor 100, and conversely to communicate data to the sensor apparatus 100 such as for firmware updates, command and control, etc.). The host platform 400 may further communicate with other devices, including e.g., a remote server or farm via a LAN/MAN/WAN 830 “cloud” infrastructure, such as via a designated URL/IP address. This cloud entity may be personalized (e.g., a networked device such as backup storage, home PC, etc.) to the sensor apparatus user, or may be accessible to/by several different users, such as that of a healthcare or service provider. In its simplest form, the host platform 400 may simply act as a receiver/display device as referenced elsewhere herein.

As shown in FIG. 3A, another exemplary system architecture 310 comprises a sensor apparatus 100 (e.g., that of FIGS. 1A-1T discussed above, or yet other types of device) associated with a user, a local receiver 400, a parent platform 700, and a network entity 830. The sensor apparatus 100 in this embodiment communicates with the local receiver 400 and/or the parent platform 700 via a wireless interface (described in detail supra) through the user's tissue boundary 101. Further, the local receiver 400 communicates (e.g., wirelessly) with the parent platforms 700 via a PAN (e.g., Bluetooth or similar) or RF interface. The parent platform 700 may also, if desired, communicate with one or more network entities 830 via a LAN/WLAN, MAN, or other topology, such as for “cloud” data storage, analysis, convenience of access at other locations/synchronization with other user platforms, etc.

In exemplary system architecture shown in FIG. 3A, the local receiver 400 is a low profile and/or wearable local receiver apparatus (e.g., small profile wristband, fob, tooth or other implant, skin-adherent patch, ear “bud” or plug, a ring worn in the finger, etc.) as compared to a dedicated receiver and processor apparatus. The local receiver apparatus 400 can include a user alert mechanism and/or minimal user interface (UI) such as, e.g., a substantially flat and flexible LED (e.g., graphene-based), AMOLED, or OTFT (organic thin-film transistor) display device, haptic mechanism (e.g., a vibration mechanism), auditory mechanism (e.g., speakers), and/or other user-signaling capabilities and mechanisms (e.g., indicator lights). Various exemplary configurations for the local receiver 400, as well as dedicated receiver and processor apparatus and parent platform apparatus, are shown and described in U.S. patent application Ser. Nos. 15/368,436, 15/645,913, and 15/853,574, each previously incorporated herein.

As indicated in FIG. 3A, the communications between the sensor 100 and the local receiver 400 and/or the parent platform 700 may be opportunistic and periodic in nature or the communications can be selectively continuous and reliable in nature (or any desired combination thereof between the various components of each). Similarly, the communication between the local receiver 400 and the parent platform can be either continuous or opportunistic. The foregoing opportunistic communication can be a significant advantage of prior art architectures; i.e., the ability for the sensor 100 to communicate periodically (as deemed necessary) with either of the reduced form-factor local receiver 400 or the parent platform 700, thereby reducing power consumption as compared to a requirement for continuous data communication with an external device. Further, such opportunistic communication enables reliable monitoring blood analyte (e.g., glucose) level, without the user being “tethered” to larger, bulkier, and perhaps activity-limiting receiving devices for extended periods of time. In such embodiments, the implanted sensor can include an internal user alert mechanism such as e.g., a haptic mechanism (e.g., a vibration mechanism), an auditory mechanism (e.g., speakers), and/or other subcutaneous user-signaling capabilities and mechanisms (e.g., indicator lights), which can be used to signal to the user that communication with an external receiver apparatus is suggested or required, thereby enabling reliable user awareness even when the external receiver apparatus is not currently within communications range of the sensor.

As indicated in FIG. 3A, the implanted sensor performs on-board processing of raw sensor data to collect and store blood analyte (BA) data. The sensor additionally performs data processing to determine whether an alert condition is present, such as e.g., determination of whether a current BA level is below a low threshold or above a high threshold, each indicating need for issuance of an alert to a user (via one or both of the receiver 400 and the parent platform 700) such that the user can take action manage their blood analyte concentration/condition.

As discussed above, the implanted sensor may include an internal user alert mechanism configured to signal to the user that there is a need to bring the sensor into communications range with an external receiving device (if not already within such a range). Upon coming into communications range, the sensor 100 opportunistically communicates with one or more of the external devices 400,700 (as necessary), such as when the sensor has new collected blood analyte data, or has determined data indicative of an alert condition, or where the external device has data for transmission to the sensor apparatus (e.g., configuration or calibration data). Exemplary methods and apparatus for determination and issuance of user alerts related to blood analyte conditions which may be utilized with the sensor apparatus 100 used in combination with one or more of the external receiver apparatus 400, 700 are described in detail with reference to FIGS. 5A-5D, as well as in U.S. patent application Ser. Nos. 15/368,436, 15/472,091, and 15/645,913, each previously incorporated herein.

In other words, in the illustrated architecture 310, the implanted sensor apparatus 100 (i) reliably operates for comparatively extended periods of time without requiring external input or calibration, or other communication; (ii) performs on-board data collection and processing; and (iii) selectively opportunistically communicates the data to the local receiver 400 (which acts as a reduced- or limited-functionality indicator and monitor for the user) and/or the parent platform 700, thereby obviating continuous communication with the external devices 400,700 and unnecessary expenditure of power source resources. Additionally, the ability of the sensor apparatus to function substantially autonomously advantageously enables the user to: (i) engage in activities which they could not otherwise engage in if “tethered” to the parent platform (or even the local receiver), and (ii) effortlessly obtain reliable blood analyte data and other useful information (e.g., trend, rate of change (ROC), and other sensor-data derived parameters), in a non-obtrusive (or even covert) manner as necessary and/or as desired.

In the example of system architecture 310, when communication between one or more of the external receiver device and the implanted sensor does occur, the exemplary architecture 310 enables two-way data transfer, including: (i) transfer of stored/accumulated sensor data to the external receiver apparatus for archiving, analysis, transfer to a network entity, etc.; (ii) transfer of sensor-specific identification data and/or receiver-specific data between the sensor and the external device; (iii) transfer of user alert/notification data; (iv) transfer of external calibration data (e.g., derived from an independent test method such as a fingerstick or blood glucose monitor and input either automatically or manually to the parent platform) from external device to the sensor apparatus; and (v) transfer of sensor configuration or other data (e.g., software/firmware updates, user-prescribed receiver settings for alarms, warning/buffer values, indication formats or parameters, historical blood analyte levels for the user, results of analysis by the local receiver 400, parent 700, and/or network entity 830 of such data, diagnoses, security or data scrambling/encryption codes or keys, etc.) from the external device to the sensor apparatus. As described in U.S. patent Ser. Nos. 15/368,436; 15/645,913; and 15/853,574, each previously incorporated herein, similar opportunistic communications and data transmissions can occur between the local receiver apparatus and the parent platform.

FIG. 3B shows yet another embodiment of a system architecture 320 for, inter alia, monitoring blood analyte levels within a user, useful with the present disclosure. As shown in FIG. 3B, the architecture 320 comprises a sensor apparatus 100 associated with a user, a local receiver 400, and a calibration sensor platform 840. As with the embodiment of FIG. 3A, the sensor apparatus 200 in this embodiment opportunistically (or continuously) communicates with the local receiver 400 via a wireless interface through the user's tissue boundary 101. The CSP 840 in the illustrated embodiment comprises a calibration data source for the local receiver 400 and/or the implanted sensor, and is configured for communication via a PAN (e.g., Bluetooth or similar), RF interface, IR (e.g., IrDA-compliant), or optical or other short-range communication modality, for transmission of calibration data. In one implementation, the CSP 840 wirelessly communicates calibration data to the receiver apparatus 400, which is subsequently transmitted from the local receiver 400 to the implanted sensor 100 via its (the sensor's) BLE interface. Advantageously, the implanted sensor 100 can communicate directly with the CSP 840 (via its BLE interface) for receipt of calibration data, thereby obviating intermediary communication with the local receiver. In other embodiments, the CSP 840 may communicate (via wireless or wired communication) with a parent platform and/or other external or internal devices.

As indicated in FIG. 3B, the communications between the sensor 100 and the local receiver 400 can be (selectively) opportunistic or continuous in nature while the communication between the CSP 840 and either of the implanted sensor 100 or the local receiver 400 is purposely opportunistic in nature. When the opportunistic communication between the CSP 840 and the implanted sensor or the local receiver does occur, the exemplary architecture 320 enables at least one-way data transfer, including transfer of external calibration data (e.g., derived from an independent test method such as the “fingerstick” or other form of blood analyte sensor 842 of the CSP 840) from the CSP to the implanted sensor or the local receiver. In an exemplary implementation, the CSP 840 comprises a “smart” fingerstick apparatus, including at least (i) sufficient onboard processing capability to generate calibration data useful with the local receiver 400 and/or the sensor apparatus 100 based on signals or data output from the blood sensor 842, and (ii) a wireless data interface to enable transmission of the data.

In one configuration, the sensor 842 includes a needle or lancet apparatus 844 which draws a sample of the user's blood for the sensor 842 to analyze. Electronic glucose “fingerstick” apparatus (including those with replaceable single-use lancets) and re-usable electronic components are well known in the relevant arts, and accordingly not described further herein. See e.g., U.S. Pat. No. 8,357,107 to Draudt, et al. issued Jan. 22, 2013 and incorporated herein by reference in its entirety, for one example of such technology. The sensor 842 analyzes the extracted blood obtained via the lancet 844 and (via the onboard processing) produces data indicative of a blood glucose level (or at least generates data from which such level may be derived), such data being provided to the communications interface 846 for transfer to the local receiver 400 and/or the implanted sensor 100. The transmitted data are then utilized within the implanted sensor or the local receiver for calibration of the blood analyte data.

In one variant, the interface 846 comprises a Bluetooth-compliant interface (e.g., BLE), such that a corresponding Bluetooth interface of the local receiver can “pair” with the CSP 840 to effect transfer of the calibration data wirelessly. Hence, the user with implanted sensor 100 can simply use a fingerstick-based or other type of external calibration data source to periodically (e.g., once weekly) confirm the accuracy and/or update the calibration of the implanted sensor 100 via opportunistic communication between the sensor and/or the local receiver and CSP when convenient for the user. Advantageously, many persons with diabetes possess such electronic fingerstick-based devices, and wireless communication capability is readily added thereto by the manufacturer at little additional cost.

In another variant, the communications interface comprises an IR or optical “LOS” interface such as one compliant with IrDA technology, such that the user need merely establish a line-of-sight path between the emitter of the CSP 840 and the receptor of the local receiver 400, akin to a television remote control. As yet another alternative, a near-field communication (NFC) antenna may be utilized to transfer data wirelessly between the apparatus 400, 840 when placed in close range (i.e., “swiped”). Yet other communication modalities will be recognized by those of ordinary skill given the present disclosure.

As described elsewhere herein, exemplary embodiments of the present disclosure may be used in combination with, inter alia, medicant delivery apparatus, such as a partially implanted, fully implanted, or non-implanted pump apparatus (or even non-pump delivery mechanisms), where the delivered medicant is intended to affect the state of a measurable physiologic parameter (e.g., blood analyte level). Notably, algorithms and mathematical models for computing an appropriate dosage of medicant may be implemented in computerized logic (software, firmware, or even hardware) that is resident in any number of different locations within the system, including: (i) within the implanted delivery (e.g., pump) apparatus itself; (ii) “off-board” the delivery apparatus, such as in an external receiver apparatus (examples of which are described below, as well as those receiver apparatus discussed with reference above with reference to the sensor system); (iii) off-board, in a data-connected “cloud” entity; (iv) off-board in a data-connected sensor apparatus; and/or (v) combinations of the foregoing (e.g., in a distributed computing architecture). Accordingly, the following embodiments are merely examples of such types of delivery system architectures, and the various aspects of the present disclosure are in no way limited thereto.

Referring now to FIG. 3C, one embodiment of a system architecture for, inter alia, monitoring blood analyte levels and communicating blood analyte levels to a medicant delivery apparatus for e.g., automatically administering medicant within a user (which may be useful with the machine learning-based methods and apparatus described in U.S. patent application Ser. No. 15/853,574, previously incorporated herein) is shown and described. As depicted in FIG. 3C, the system architecture 1100 comprises a sensor apparatus 100 (e.g., that of FIGS. 1A-IT discussed above, or yet other types of sensor devices) associated with a user via subcutaneous implantation, a sensor local receiver apparatus 400 selectively in opportunistic or continuous data communication with the sensor apparatus 100 and/or a parent platform 700, a transcutaneous pump and processor apparatus 1000 in opportunistic or continuous data communication with the implanted sensor 100 (and/or the local receiver apparatus 400), which is associated with the user via transcutaneous implantation of at least a portion of the apparatus (e.g., transcutaneous insertion of an insertion set or a cannula through the tissue boundary 101).

In the system architecture 1100, the pump apparatus and pump receiver/processor apparatus 1000 comprise an integrated pump device, and thus a wireless interface of the integrated device is used primarily for communication via PAN (e.g., BLE) (or alternatively WLAN, narrowband, RF, or other communication modality) with the sensor 100 and/or the local receiver 400 for receipt of blood analyte data and/or medicant dosage data.

It is also appreciated that other forms of wireless communication may be used for such applications, including for example inductive (electromagnetic induction) based systems, those based on capacitance or electric fields, or even optical (e.g., infrared) systems where a sufficiently clear path of transmission and reception exists, such as two devices in immediately adjacent disposition, or ultrasonic systems where the two devices are sufficiently close and connected by sound-conductive media such as body tissues or fluids, or a purposely implanted component. Hence, the following embodiments are merely illustrative.

As discussed with regard to the embodiments of FIG. 3A and 3B, the sensor apparatus 100 communicates (via e.g., PAN such as BLE, 802.15.4 or Z-Wave narrowband, or in alternate embodiments via RF such as at 433 MHz) with the receiver/ processor apparatus 400 via a wireless interface (described in detail supra) through the user's tissue boundary 101. In the present embodiment, each of the sensor 100, the sensor receiver/processor apparatus 450, and the pump and processor apparatus 1000 may also, if desired, opportunistically or continuously communicate with one or more network entities 830 (such as for cloud data storage, analysis, convenience of access at other locations/synchronization with other user platforms, etc.) via a parent platform 700 of the type previously described herein (e.g., via a PAN or LAN connection to the parent device 700, the latter which interfaces with a LAN/WLAN, WAN, MAN, or other communication modality, or directly with the network entities via the aforementioned protocols.

As shown in FIG. 3D, yet another exemplary system architecture 1120 comprises a sensor apparatus 100 (e.g., that of FIGS. 1A-1T discussed above, or yet other types of sensor devices) associated with a user via subcutaneous implantation, a sensor receiver/processor apparatus 400 in opportunistic or continuous data communication with the sensor apparatus 100, a fully implanted pump apparatus 1020a associated with the user via subcutaneous implantation, a pump receiver/processor apparatus 1020 in continuous data communication with the sensor receiver/ processor apparatus 450 and also in continuous or opportunistic data communication with the fully implanted pump apparatus 1020a, and a network entity 800.

In the exemplary system architecture shown in FIG. 3D, the fully implanted pump apparatus 1020a is a subcutaneous pump apparatus implanted beneath the user's skin. Similar to the implanted sensor, in some examples, the fully implanted pump apparatus 1020a can include a user alert mechanism such as e.g., a haptic mechanism (e.g., a vibration mechanism), an auditory mechanism (e.g., speakers), and/or other subcutaneous user-signaling capabilities and mechanisms (e.g., indicator lights).

The sensor apparatus 100 communicates (via e.g., PAN such as BLE of narrowband RF) with the local receiver/processor apparatus 400 via a wireless interface (described in detail supra) through the user's tissue boundary 101. In the system architecture 1120, the pump apparatus and receiver/processor apparatus 1020 comprises a non-integrated device, and thus a wireless interface of the sensor apparatus 100 is used for (i) opportunistic or continuous communication via PAN, LAN, RF, or other communication modality with the local sensor receiver/processor 400 for receipt of blood analyte data, as well as (ii) opportunistic of continuous communication with the fully implanted pump apparatus 1020a for transmission of blood analyte data and/or medicant dose data, and receipt of pump data (e.g., data related to reservoir levels, pump firing, pump stroke volume, etc.).

Each of the implanted sensor 100, the local receiver/processor apparatus 400, the implanted pump 1020a, and the pump receiver/processor apparatus 1020 may also, if desired, opportunistically or continuously communicate with one or more network entities 800 via the parent platform 700, such as for cloud data storage, analysis, convenience of access at other locations/synchronization with other user platforms, etc.

It will be appreciated that the implanted pump 1020a and sensor 100 may be integrated at least with respect to their (external) communication modality, such that a single wireless interface is used to communicate with an external receiver (such as for example an integrated sensor/pump/parent processor). In one variant, the interface comprises a PAN (e.g., BLE or 802.15.4) interface, such the receiver/processor apparatus merely need be within a certain range of the user for communication of blood analyte data and/or pump data.

Additional functionalities of the local receiver 400, parent platform 700, a dedicated receiver/processor apparatus (for the sensor and/or the pump), and various pump apparatus and other medicant delivery mechanisms, as well as various system architecture and communication pathways of system components which may be useful with the system architectures of FIGS. 3A-3D are described in U.S. patent application Ser. Nos. 15/368,436, 15/645,913, and 15/853,574, each previously incorporated herein.

Further, it will be appreciated that the architectures shown in FIGS. 3A-3D (as well as those described in U.S. patent application Ser. Nos. 15/368,436, 15/645,913, and 15/853,574, each previously incorporated herein) are in no way exclusive of one another, and in fact may be used together (such as at different times and/or via different use cases), such as in the examples described above. Myriad other permutations of use cases involving one or more of the various described components are envisaged by the present disclosure.

FIGS. 4A-4D are functional block diagrams illustrating exemplary embodiments of the exemplary implantable sensor apparatus 100, and devices configured for data communication with the implanted sensor apparatus, such as the sensor receiver apparatus 400, the pump processor apparatus 1000, the pump receiver/processor apparatus 1020, and the implanted pump 1020a shown in FIGS. 3A-3B herein.

These and additional configurations and functionality of the sensor apparatus are described in detail U.S. patent application Ser. Nos. 15/359,406, 15/368,436, 15/645,913 and 15/853,574, each previously incorporated herein.

### Sensor System Operational Methods

Referring now to FIGS. 5A-5D, exemplary embodiments of the methods of operating the analyte sensing system (e.g., a system including the implanted sensor 100 and a receiver apparatus, such as the receiver apparatus 400, the parent platform 700, and/or another receiving device) are described in detail.

FIG. 5A is a logical flow diagram depicting an exemplary embodiment of a generalized method 500 for operation of the implantable sensor apparatus (within a sensor system including an external receiver apparatus) according to the present disclosure. As shown in FIG. 5A, the method 500 includes first enabling and implanting the sensor 100 (or others) per step 502. In one embodiment, the sensor is enabled, implanted in the host (such as via the procedures described in U.S. patent application Ser. No. 14/982,346, previously incorporated herein), and tested as part of step 502.

Next, the receiver apparatus (e.g., any of those of FIGS. 3-3D and 4B-4D herein, or another receiver apparatus) is optionally enabled, per step 504. In one implementation, the receiver apparatus is maintained within communications range of the sensor apparatus. In another implementation, the receiver apparatus is enabled and functionally tested to ensure the sensor and receiver are communication capable, and subsequently the receiver is brought into communications range by the user periodically (e.g., at regular intervals, as desired by the user based on “feeling” or observation of their disease symptoms, or in response to an internal alert generated by the sensor). In either implementation, data communication and/or communication pairing between the implanted sensor and the receiver apparatus may be opportunistic (discussed in detail infra). Alternatively, in the implementation where the receiver apparatus is maintained within communication range, data communication and pairing between the implanted sensor and the receiver apparatus may be continuous (if desired, such as e.g., for a patient whose particular disease presentation is severe and requires continuous monitoring).

It will be appreciated that in some implementations, presence of/communication with the receiver is not a predicate for setup/operation of the sensor apparatus. For example, in one variant, the sensor apparatus, after confirming proper operation of its internal functions, may in fact operate autonomously for a period of time before stablishing any communication with any external device, and may e.g., simply generate, process, and store data for later transmission or “download” to the receiver when the latter becomes available. Moreover, in some cases (e.g., where communication of data to the user is haptic or otherwise not reliant on an external receiver per se), the sensor apparatus 100 may operate independently of any external receiver, at least for period of time.

Optionally, also at step 504, one or more other devices which are configured for use with the implanted sensor (such as any of those shown in FIGS. 3B-3D and 4C-4D) may be enabled and brought into communications range with the sensor. For example, a pump apparatus may be enabled and implanted (partially or fully), a non-implantable pump apparatus may be enabled, and/or a computerized calibration device may be enabled. The enabled device can be brought into communications range with the implanted sensor and either functionally tested and then periodically brought in into communications range with the sensor or maintained within communications range throughout its use. Alternatively, such devices can be within range continuously, and merely be switched on/off in terms of being discoverable by the sensor apparatus, and/or initiating probe, advertisement, or handshake protocols to attempt to advertise their presence and availability for a data communications session.

In one specific implementation, a pump and processor apparatus is powered on, tested, calibrated, user preference or medicant dosing settings entered, reservoir filled, etc., and an insertion set or cannula (which is fluidly coupled to a reservoir within a housing of the apparatus via a catheter) is implanted transcutaneously through the skin of a user and attached at the insertion site. In yet another implementation, the fully implantable pump apparatus is enabled (e.g., powered on, calibrated, reservoir filled, etc.) and surgically implanted within the user subcutaneously and the medicant-dispensing cannula associated therewith directed into a body cavity (e.g., within the intraperitoneal cavity). In one specific example, the fully implantable pump apparatus is a separate device from the sensor that is implanted either in a same (contemporaneous) surgical procedure or in a different (non-contemporaneous) surgical procedure at a distinct implantation site from the sensor. In an alternate example, the fully implantable pump apparatus is integrated with the sensor, and is co-enabled and co-implanted as a single device at a single implantation site.

In each of the foregoing implementations, the sensor apparatus 100 includes a Bluetooth wireless interface (e.g., BLE variant) which operates at 2.4 GHz and which has been demonstrated by the Assignee hereof to penetrate human tissue with sufficient efficacy so as to maintain a wireless communication channel between e.g., the implanted sensor apparatus and the comparably Bluetooth-equipped device (e.g., a local receiver, a parent platform, a dedicated receiver/processor, an implanted pump apparatus or pump receiver, a non-implanted pump apparatus, a calibration device) the latter further including an application program or firmware configured to extract data (whether raw or on-board pre-processed or fully processed data) from one or more messages wirelessly transmitted from the sensor. As discussed supra with respect to FIGS. 2D-2J, the communications range of the implanted sensor may vary depending upon, inter alia, the physical attributes of the user within which the sensor is implanted, the location of implantation, the environment within which the sensor and communicative device are utilized (e.g., the presence of reflective surfaces within the environment, etc.), and/or an orientation of the communicative device (e.g., upright vs. laying down). Generally, communications range is functionally defined as within a room where the user (having the implanted sensor) is located or within approximately 3-6 m of the implanted sensor when the user is outdoors.

Subsequent to enablement and implantation of the sensor (and enablement of the receiver), the sensor system may be operated in an initial sensor “training mode,” wherein the detector elements of the sensor 100 are operational and producing signals, yet the data are not output to the user or other entity, but rather used for “off line” analysis and error model generation. Data collected and/or received during the sensor training mode operation are then used to (i) calculate sensor error (via e.g., comparison of sensor blood analyte data to externally collected reference blood analyte data), and (ii) generate a sensor operational model (such as e.g., a user-specific sensor operational model).

In one implementation, time-stamped sensor calculated blood analyte data are collected, and are analyzed via comparison to time-stamped blood analyte reference data collected contemporaneously with the sensor data to determine sensor error data. Additionally, other data are collected and stored (e.g., data from one or more other sensors (such as temperature data, motion data, orientation data, pulse rate data, other blood analyte concentration data, manually entered user data, etc.), time of day, blood analyte level range, etc.). Data collected and/or received during the sensor training mode operation are then used to generate and store a sensor operational model (such as e.g., a user-specific operational model for operation of the particular sensor used by that individual). Sensor training mode operation and operational model generation useful for compensating for unmodeled system error after implantation of the sensor and which can improve sensor accuracy (such as improvement of accuracy of the implanted sensor 100 disclosed herein) are discussed in detail in U.S. patent application Ser. Nos. 15/645,913 and 15/853,574, each previously incorporated herein.

In the present embodiment, the “machine learning” aspects utilized in sensor operational model generation are indigenously stored at and employed on the implanted sensor apparatus 100 itself, thereby effectively obviating the need for communication with the corresponding receiver/processor apparatus, at least for functions relating to systemic or other error modeling and correction. Alternatively, sensor operational model generation (or discrete portions thereof) may be carried out by the receiver/processor device, such generated models or components thereof which can be stored at the receiver/processor and/or later transmitted to the implanted sensor for storage and implementation thereon, such as via firmware or software update, and/or data upload. Sensor “re-training” and generation of a new (updated) operation model can be carried out as desired and/or if one or more re-training criteria are met (e.g., at expiration of a pre-determined time period, detection of increased error, detection of an event, such as an impact event or a high temperature event). Alternatively, operation of the sensor system utilizing the initial sensor operational model is continued until explant of the sensor.

Similarly, subsequent to enablement and implantation of the pump apparatus (and enablement of any corresponding receiver/processor for the exemplary pump apparatus), the sensor and pump system can be operated in an initial “pump training mode,” wherein corrected blood analyte data are received from the sensor 100, and are utilized to calculate medicant dosage for administration from the pump based on an initial dosing calculation algorithm. In one implementation, time-stamped blood analyte data are collected before, during, and/or after medicant delivery, and are analyzed via comparison to an expected outcome or BA target data (e.g., an expected response curve or a table of data corresponding to an expected response curve) to determine pump error data. Additionally, other data are collected and stored (e.g., data from one or more other sensors (such as temperature data, motion data, orientation data, pulse rate data, other blood analyte concentration data, manually entered user data, etc.), time of day, blood analyte level range, etc.). Data collected and/or received during the pump training mode operation are then used to generate and store a pump operational model (such as e.g., a user-specific operational model for operation of the particular pump or other delivery device used by that individual). It will be appreciated that in alternate embodiments, sensor and pump training can be carried out simultaneously, and/or pump training can be carried out without use of a “trained” sensor by carrying out pump training with receipt of externally generated blood analyte reference data. Pump training mode operation and operational model generation useful for compensating for unmodeled system error after implantation of the sensor and pump, and which can improve accuracy (and efficacy) of medicant delivery are discussed in detail in U.S. patent application Ser. No. 15/853,574, previously incorporated herein.

Additionally, the “machine learning” aspects utilized in pump operational model generation (if present, and as contrasted to the sensor model(s) described supra) may be indigenously stored and employed on the implanted sensor apparatus, thereby effectively obviating the need for communication with a corresponding receiver/processor apparatus, at least for functions relating to systemic or other error modeling and correction. Alternatively, pump operational model generation may be carried out (in whole or part) by the receiver/processor device, which can be stored at the receiver/processor and/or transmitted to the implanted sensor or the implanted pump for storage and implementation thereon. Pump “re-training” and generation of a new (updated) operation model can be carried out as desired and/or if one or more re-training criteria are met (e.g., at expiration of a pre-determined time period, detection of increased error, detection of an event, such as an impact event or a high temperature event).

It is also appreciated that while the generalized methodologies set forth above utilize implant of the sensor apparatus and/or pump apparatus as a precondition for training of the machine learning algorithms (so as to ostensibly provide the best training environment for that particular sensor/user combination), this may not always be a requirement. For example, the present disclosure contemplates conditions where the sensor and/or the pump may be “pre-trained” prior to implantation, such as based on data previously acquired for that same individual (e.g., as part of a prior training session and/or prior sensor implantation), or even data derived from one or more similarly situated individuals (e.g., family member, similar physiologic characteristics, similar disease expression, etc.). In such cases, the sensor and/or pump implanted in the individual may for instance be pre-programmed with data representative of a prior sensor and/or pump operational models using wireless or other data communication (such as via Bluetooth BLE wireless interface(s) described supra) prior to implantation, such that the sensor and/or pump operational model(s) (data) are stored and accessible immediately upon activation of the sensor and/or pump in vivo.

Returning to FIG. 5A, after generation of any sensor and/or pump operational models, the sensor system is operated in a detection mode (i.e., a mode whereby analyte data collected from the user are corrected as needed, and output for use by the user or other entity such as a caregiver, or for use by other communicative devices such as a communicative (non-implanted or implanted) pump). Specifically, per step 506, sensor element signals (e.g., raw sensor data) are collected by the on-board processor and stored thereon. At step 508, the sensor performs on-board processing of the sensor signals/data to determine blood analyte levels and/or related parameters (e.g., rate of change (ROC), other trend data, or identification of one or more alert conditions related to the blood analyte level).

Based at least in part on the availability of the processed blood analyte data, the sensor then may optionally determine communication enablement status per step 512. If enabled, the sensor 100 may then for example generate and periodically transmits “beacon” data therefrom (e.g., a UUID or other data structure via its BLE communication interface). It is appreciated that while FIG. 5 shows the generation of the “beacon” signal responsive to the determination of step 512 (i.e., whether wireless communication is enabled, such as via determining whether a viable communication session between the sensor PHY and that of an external device is extant), other criteria may be used including e.g., the presence of calculated (i.e., ready for transmission) blood analyte and/or alert data, as may a simple “store and transmit later” protocol as described elsewhere herein.

Turning to FIG. 5B, an exemplary embodiment of a method 511 of data processing and output, during detection mode operation of the implanted sensor 100, is now described in detail. Specifically, the method 511 includes first receiving detection signals from the sensor elements (i.e., one or more working electrodes each associated with one or more reference electrodes) at step 515. Further, the received signals are processed, and the processed and/or unprocessed (raw) data stored.

Next, per step 517, a blood analyte level is calculated. At minimum, “calibrated” BA data (BA_{cal}) is computed by applying the known calibration transform to the raw sensor data (or processed sensor data) to provide a closer approximation of an actual blood analyte level (albeit still containing effects due to random noise, one or more unmodeled variables, temporal mismatch between working and reference electrodes, etc.).

Optionally, the BA_{cal} data may be further processed via application of one or more stored sensor operational models on (i) the BA_{cal} data, and (ii) data from the one or more identified parameters which are correlated with sensor error (BA_{s_error}) (such as e.g., temperature data, motion data, orientation data, pulse rate data, other blood analyte concentration data, manually entered user data, etc.), thereby generating BA_{cal} data corrected for BA_{s_error} (i.e., systemic error from unmodeled user-specific variables). In one implementation (implanted blood glucose sensor), once a new blood glucose sample is recorded by the system, it will compute all the model parameters selected and defined in the model parameters identification process using the new BG sample data (and any number of past samples needed). Once the model parameters are computed, the machine-learned sensor operation model is applied to predict the BG_{s_error}, and the predicted BA_{s_error} is subtracted from BA_{cal}.

Further, one or more random noise signal filters can optionally be applied to the corrected BA_{cal} reading to additionally correct for error due to random noise (“e”). For example, one or more random noise signal filters (such as e.g., finite impulse response (FIR), infinite impulse response (IIF), Kalman, Bayesian, and/or other signal processing filters) can be applied to the BA_{cal} reading to correct for error due to random noise. As a brief aside, as will be appreciated by artisans of ordinary skill in the signal processing arts, typical “white” noise or “random” noise is characterized by a constant power spectral density. Colored noise spectra may have non-constant power spectral density; e.g., red tinted noise has less attenuation at longer wavelengths (lower frequencies), whereas blue tinted noise has less attenuation at shorter wavelengths (higher frequencies). Common techniques for removing the effects of random noise include without limitation e.g., time based averaging, statistical sampling, spectrally weighted averaging, and/or any number of other weighted filtering techniques.

Yet further, in addition to application of the sensor operational model(s) and random noise filters discussed supra, the sensor 100 may utilize one or more algorithms for correction of temporal mismatch between paired and/or grouped reference and working electrodes of the sensor. Specifically, in one implementation, a model is utilized that establishes the differences in the time responses of two electrodes (i.e., of the aforementioned pair) as a function of the ratio of analyte (e.g., glucose) concentration to background reaction product (e.g., oxygen) concentration. Moreover, given that the time constants (and/or delay) of the two electrodes are predictable (can be estimated) at any given time, correction or compensation for the temporal response (lag and/or delay) mismatch between the two electrodes can be computed, and thereby enable more accurate computation of the target analyte such as glucose, through the exemplary differential (ratiometric) electrodes. Detailed descriptions of temporal mismatch correction models and algorithms which can be utilized with the sensor apparatus 100 are disclosed in U.S. patent application Ser. No. 16/233,536 filed Dec. 27, 2018, previously incorporated herein.

At step 519, other parameters of interest if any (such as real-time trend and/or rate of change (ROC)) can be calculated based on the corrected BA_{cal} data. Based on the corrected BA_{cal} data and/or other parameters of interest, it is next determined if one or more disease alert conditions are present (within the user) at step 521. Specifically, after a current blood analyte level and/or other blood analyte parameters are determined, the data is then compared to specified blood analyte thresholds. In one implementation, the foregoing thresholds comprise user-specified, medical professional-specified thresholds, or pre-determined thresholds (e.g., a low blood analyte threshold or a high blood analyte threshold) that are indicative of a medical condition which requires a user action in order to manage the blood analyte level and/or indicative of a blood analyte level approaching such a condition. If such a condition is present, per step 523, UUID or other advertisement data is generated such that it is indicative of the identified alert condition.

In one variant, user-specified or medical professional-specified parameters for operation of the blood analyte detection system include an Ideal Range (IR), and high and low

Buffer Zone Ranges (BZRs) for blood analyte level. This approach enables definition of a Priority Range (PR); i.e., a blood analyte level that lies outside of the high and low BZRs. The BZRs may enable provision of pre-alert notifications (i.e., progressive “soft” alert notifications) via the UI as the blood analyte level approaches a level of greater concern within the PR. Accordingly, if the blood analyte level is greater than the high threshold of the high

BZR (i.e., within the PR high range) or less than the low threshold of the low BZR (i.e., within the PR low range), a priority alert notification is generated; however, if the blood analyte level is within the BZR, a soft alert notification is generated. Alternatively, if the blood analyte level is within the IR, no alert notification is generated. Additional details regarding alert mechanisms and strategies which can be implemented in the logic of the implanted sensor apparatus 100 (and/or its communicative receiver apparatus) are disclosed in U.S. patent application Ser. No. 15/472,091 filed on Mar. 28, 2017, previously incorporated herein.

Additionally at step 521, the sensor apparatus can determine whether calibration is needed (e.g., derived from an independent test method such as a fingerstick or blood glucose monitor and input either automatically or manually). For example, the sensor apparatus may be pre-programmed to request calibration data at pre-determined intervals (e.g., weekly) and/or the sensor apparatus may be configured to detect data errors (based on e.g., a number of data outliers being greater than a pre-determined threshold).

At step 525, it is additionally determined whether the sensor apparatus 100 includes new blood analyte data. In one implementation, the sensor apparatus may evaluate a time of last data upload to a receiver apparatus against one or more time-stamps associated with blood analyte data in order to determine/identify the presence of new data (i.e., data having a time-stamp that is at a later time than a time of the last data upload). In another implementation, any new data received after a last data upload is tagged as new data, and the tag is removed or altered after data transmission off-sensor. In yet another implementation, data is automatically deleted after transmission to a receiver apparatus, and therefore any data stored at the sensor apparatus comprises new data. It will be appreciated that the foregoing implementations are merely exemplary and various other techniques may be used to label and/or identify “new” data. Per step 527, if it is determined that new BA data is stored on the sensor, the beacon data is generated such that it is indicative of the new data.

Alternatively, if it is determined that no alert condition is present, no calibration is needed, and no new data is present, the UUID or other advertisement data is generated to indicate to the receiving apparatus that there is no need for communication/pairing required for operation of the sensor apparatus (step 529). In any of the above scenarios, the generated advertisement data is then transmitted from the sensor apparatus via its wireless communication interface (BLE interface) according to a prescribed periodic transmission window (e.g., a 10 second transmission window).

It will be appreciated that the advertisement data comprises a relatively small transmission payload (e.g., only a few bytes) utilized for determination of connectivity with a receiving device such as any of those disclosed herein. Hence, limited electrical power is consumed in transmitting such advertisements. It will also be appreciated that in other embodiments, a carrier sense or other such mechanism may be used by a putative receiver device to determine an absence of an advertisement or beacon requiring alert/blood analyte data transmission. For instance, in one variant, the absence of any signal within a prescribed time slot and/or frequency band (e.g., a BLE advertisement band of FIG. 2K-1) is used as an indication to the receiver that no alert/transmission is present or required. For instance, RSSI or other signal/energy measurements can be used to sense the presence of signals in the prescribed frequency/time window; if no carrier or signal is present, it is assumed no data need be transmitted, and/or no alert is present.

It will be appreciated that in embodiments where the implanted sensor includes a user output apparatus 188, the determinations described above to generate beacon data can be used to determine the need for generating an alert to the user via the user output apparatus. In one implementation, if an alert condition is present, calibration is needed, and/or new data is present, the sensor apparatus will generate the appropriate beacon data and will additionally generate a corresponding command for a user notification via the output apparatus (e.g., a triggering of a haptic or auditory mechanism) to alert the user that a receiving apparatus should be brought into communications range with the implanted sensor. In one variant, the user alert is trigged only after (i) transmission of advertisement data indicating a need for communication (e.g., data indicative of an alert condition present, calibration needed, and/or new data present, or merely the presence of the advertisement transmission itself), (ii) a prescribed delay period has lapsed, and (iii) no pairing with an external receiving device has occurred. In other words, in the foregoing variant, the user output apparatus is utilized/activated only in conditions where the sensor requires communication and no appropriate external receiving apparatus is communicative (e.g., powered on and within communications range), thereby conserving power resources of the implanted sensor.

Returning again to FIG. 5A, per step 512, it is determined whether communication with a receiving device has been enabled (discussed infra with reference to FIGS. 5C and 5D). If no communication is enabled, normal operation of the sensor apparatus (i.e., receiving and processing of sensor data, and generating and transmitting beacon data) is continued. However, if communication is enabled (as determined by the receiving device, see e.g., FIG. 5D), the sensor apparatus is communicatively paired with the receiving device at step 514. In one implementation, the sensor apparatus and the receiving device are paired according to a PAN protocol such as BLE, discussed supra.

In some variants, the pairing is according to a secure protocol (e.g., one requiring authentication and also utilizing encryption of various transacted data). In some examples, security is ensured by requiring close-proximity pairing (i.e., a requirement that the external device is within close physical range of the implanted sensor apparatus) via lowering of the transmission power levels and/or requiring a secondary physical interaction, such as an NFC connection or activation of the magnetic reed switch. Additionally or alternatively, enablement of communication between the sensor and an external device can utilize other two-factor authentication schemes, such password or serial number-protection, thereby requiring entering or registration of a hard-coded or encrypted sensor serial number into the external device or another device (e.g., an associated computer) during the pairing procedure.

Specifically, in the exemplary Bluetooth context, authentication and key derivation are performed according to prescribed algorithms. Bluetooth key generation is generally based on a Bluetooth PIN, which must be entered into both devices (e.g., the implanted sensor before implantation, and the receiving apparatus), or one of the devices may have a fixed PIN (such as e.g., fully implanted devices having no or limited UI or being implanted). During pairing, an initialization key or master key (i.e., private shared key) is generated, using a specified algorithm. A cipher is used for encrypting packets, granting confidentiality, and is based on a shared cryptographic generated link key or master key. The keys are used for subsequent encryption of data sent via the wireless interfaces of the paired devices and rely on the Bluetooth PIN, which has been entered into one or both devices. The foregoing pairing protocols are described in detail in Bluetooth Specifications “Multi-Channel Adaptation Protocol” published (revised) Jan. 24, 2007 and “Bluetooth Compliance Requirements” published Dec. 6, 2016, each of which is incorporated herein by reference in its entirety.

After pairing and establishment of data communication between the implanted sensor and the receiving apparatus, calculated blood analyte levels, other determined parameters of interest (e.g., ROC), and/or other data (e.g., raw/unprocessed analyte sensor data, data from other on-board sensors such as temperature, pressure, and/or motion data, etc.) are transmitted from the implanted sensor to the receiving apparatus, per step 516. In a condition where the implanted sensor has no alert conditions or new sensor data to communicate (and pairing/communication is based on a determination by the receiving apparatus that there is a need to transmit data, such as calibration or configuration data, to the implanted sensor, discussed infra), step 516 may be obviated.

Next, per steps 518 and 522, the sensor apparatus identifies whether new calibration data and/or configuration data (such as e.g., a user command or input) are received from the paired receiving device. If so, such calibration and configuration data are implemented by the sensor apparatus at step 520 and 524.

FIG. 5C is a logical flow diagram depicting an exemplary embodiment of a generalized method 530 for operation a receiving apparatus (such as the local receiver 400, the parent platform 700, or other) within the sensor system according to the present disclosure. As shown in FIG. 5C, similar to method 500, the method 530 includes first enabling and implanting the sensor 100 (or others) per step 502, and enabling the receiver apparatus (e.g., any of those of FIGS. 3A-3D and 4B-4D herein, or another receiver apparatus) per step 504.

As discussed supra with reference to method 500, in one implementation, the receiver apparatus is maintained within communications range of the sensor apparatus (enabling continuous or opportunistic data communication with the sensor). In another implementation, the receiver apparatus is enabled and functionally tested to ensure the sensor and receiver are communication capable, and subsequently the receiver is brought into communications range by the user periodically (e.g., at regular intervals, as desired by the user based on “feeling” or observation of their disease symptoms, or in response to an internal alert generated by the sensor), thereby enabling opportunistic data communication of the devices and freedom for the user from being tethered to the receiver apparatus. Optionally, also at step 504, one or more other devices which are configured for use with the implanted sensor (such as any of those shown in FIGS. 3B-3D and 4C-4D) may be enabled and brought into communications range with the sensor. For example, a pump apparatus may be enabled and implanted (partially or fully), a non-implantable pump apparatus may be enabled (as described above), and/or a computerized calibration device may be enabled. The enabled device can be brought into communications range with the implanted sensor and either functionally tested or maintained within communications range throughout its use.

Similar to the sensor 100, the receiver apparatus includes a Bluetooth wireless interface (e.g., BLE variant) which operates at 2.4 GHz, and which has been demonstrated by the Assignee hereof to have sufficient sensitivity to the transmitted energy from the sensor which penetrates the tissue of the host with sufficient efficacy so as to maintain a wireless communication channel between the implanted sensor apparatus and receiving device (e.g., a local receiver, host or parent platform, a dedicated receiver/processor, an implanted pump apparatus or pump receiver, a non-implanted pump apparatus, a calibration device) the latter further including an application program or firmware configured to extract data (whether raw or on-board pre-processed or fully processed data) from one or more messages wirelessly transmitted from the sensor apparatus 100.

In one implementation, the GUI of the receiving apparatus is configured to indicate to a user when it is within communications range of the implanted sensor. For example, FIGS. 6A and 6B show “blinded” graphical presentations 602 and 630 for receiving devices, which indicate that no sensor is detected. Alternatively, other graphical presentations (such as graphical presentations 604-616, 618-628 and 632) indicate that the sensor is within communications range. As noted supra, the device may be within a range where wireless session/channel establishment is possible, but with one or other device being in a non-communicative mode (e.g., the sensor in a “sleep” or non-transmissive mode due to no pending data or alerts, or the receiver being in a sleep or other mode and not awoken by an advertisement or beacon from the sensor).

During “detection mode” operation of the sensor apparatus (subsequent to any “training mode” operation of the sensor apparatus and/or an implanted pump apparatus, discussed supra and in U.S. patent application Ser. No. 15/645,913 filed Jul. 10, 2017 and Ser. No. 15/853,574 filed Dec. 22, 2017, each incorporated by reference herein), the sensor 100 may decide, via onboard logic, that communication with the receiver is needed, and hence issue advertisement or beacon data which can be discovered by the receiver per step 534); the receiving apparatus is then communicatively paired with the implanted sensor (step 536), and data is received from and/or transmitted to the sensor (step 538).

As can be seen in FIG. 5D, an exemplary embodiment of a method 537 for establishing communication and sharing data with the implanted sensor includes first enabling reception of or scanning for the sensor advertisement or beacon, such as for a duration of a scan window or using other protocols as described previously herein (step 539). Next, at step 541, the advertisement/beacon signal is received on at least one frequency channel (e.g., at least one of the three BLE frequency channels discussed supra with reference to FIG. 2K). Per step 543, the advertisement data is analyzed by the receiving apparatus to determine whether it is indicative of the presence of new blood analyte data (e.g., data that has not been previously transmitted from the sensor apparatus), the presence of an alert condition (e.g., a blood analyte level of the user is above or below predetermined thresholds), and/or a need for calibration of the sensor (e.g., a request from the sensor apparatus for external calibration data).

Per step 545, the receiving apparatus additionally determines whether it has data (stored thereon) data which should be transmitted to the implanted sensor apparatus, such as new configuration or user input data, calibration data, data from other external sensors, and/or other data.

As indicated at step 547, if it is determined that (i) the advertisement data (or lack of an advertisement) lacks any of the foregoing indicators of a need for transmission of data to or from the sensor, and (ii) the receiving apparatus does not require transmission of data to the sensor, then no pairing of the devices occurs (or any existing session is terminated). It will be appreciated that although communication is stopped, the receiving apparatus will continue to monitor for new advertisement or beacon data according to its programmed logic (e.g., at prescribed temporal intervals within the prescribed advertising frequency bands). Alternatively, at step 549, if it is determined that (i) the advertisement or beacon data includes any of the foregoing indicators of a need for transmission of data to or from the sensor, and/or (ii) the receiving apparatus determines requirement for transmission of data to the sensor, then paring of the devices occurs if not already established.

The receiver apparatus then wirelessly transmits and/or receives (optionally) encrypted data at prescribed intervals or according to a preset protocol at step 551. Any encrypted data received at the receiving apparatus is validated and decrypted utilizing the foregoing private shared key (step 553), and the decrypted data is optionally stored and time-stamped at the receiving device (step 555). Exemplary implementations of sensor data receipt and demodulation/unscrambling methodology for the receiver apparatus are further described in U.S. patent application Ser. No. 15/368,436 previously incorporated herein.

It will be appreciated that any calibration and/or configuration data or changes pushed to the sensor apparatus may also be encrypted or otherwise protected, such as to frustrate surreptitious provision of data to the sensor which could interrupt or corrupt its operation. As such, mutual authentication is also optionally utilized such that the sensor authenticates the receiver, and the receiver authenticates the sensor, during pairing or other data session establishment. Physical layer security (e.g., via FHSS hopping code or DSSS long code secrecy) or other mechanisms may also be utilized to frustrate such surreptitious activity. One-way cryptographic hashes may also be applied to data transmitted in either direction.

In one implementation, the data received from the sensor 100 by the external receiver is further transferred to another receiving device (such as transfer of received sensor data from the local receiver apparatus 400 to a parent platform 700), and/or the data is yet further transferred to a network server or storage location (such as e.g., transfer of the received sensor data from the receiver 400 or parent platform 700 to the network entity 830).

Returning to FIG. 5C, per step 540, the blood analyte level (e.g., corrected BA_{cal} data) and/or other parameters of interest if any (such as real-time trend and/or ROC data) are converted to a prescribed output format (e.g., a graphic rendering of a numeric value, a graphic display of a trend arrow, a sequence of haptic vibrations, etc.) consistent with the selected/configured output modality. The converted values or indications can then be output to the user in the appropriate modality/modalities per step 542, such as via the GUI or other user output mechanism associated with the receiver apparatus. As can be seen in FIGS. 6A and 6B, graphical presentations 604 and 620 show exemplary output of blood analyte level and trend information based on data received from the sensor. Additional graphical presentations of blood analyte data output which are usable in the implanted sensor system of the present application are shown and described in U.S. patent application Ser. No. 15/472,091, previously incorporated herein.

Additionally, the method 530 further includes determining whether the received data comprises a BA alert condition (step 544), and, if so, generating an appropriate user alert indicative of the alert condition (step 546). In one implementation, step 546 further includes receiving user input (input via its GUI or a GUI of another user input device in data communication therewith) in response to the user alert, such as data indicative that a user action has been taken (e.g., ingestion of slow or fast-acting carbohydrates, intake of medicant, etc.). Graphical presentations of user alerts based on blood analyte data which are usable in the implanted sensor system of the present application are shown and described in U.S. patent application Ser. No. 15/472,091, previously incorporated herein.

The method 530 yet further includes identifying data indicative of a requirement for calibration of the sensor apparatus (step 548). In one implementation, the receiver apparatus may identify that its own data storage apparatus has new calibration data stored thereon. In another implementation, the receiver apparatus may identify a request for calibration data received from the sensor. In the latter implementation, per step 550, the receiver apparatus may (in response) generate a request for calibration data. In one variant, the request for calibration data comprises generating a user alert configured to indicate to a user that calibration is needed, and that the user should manually enter BA level reading or utilize a computerized calibration device (such as e.g., CSP 840). In another variant, the request for calibration data comprises generating and wirelessly transmitting a request for calibration data directly to the computerized calibration device. If the calibration device has the requested calibration data stored thereon, it may be automatically transmitted to the receiver apparatus. Otherwise, the calibration device may additionally (or alternatively) generate a user notification to take action for calibration (e.g., take a “fingerstick” blood sample). Accordingly, step 550 may further include receiving calibration data. Examples of calibration alert outputs and user calibration input mechanisms are shown in graphical presentations 606-608, 612-614, 622 and 626-628 of FIGS. 6A and 6B. Additional graphical presentations of calibration alert outputs and user calibration input mechanisms which are usable in the implanted sensor system of the present application are shown and described in U.S. patent application Ser. No. 15/472,091, previously incorporated herein.

Per step 552, the exemplary method still further includes determining a presence of received user command data (input via its GUI or a GUI of another user input device in data communication therewith), which may be stored at the receiver apparatus (i.e., entered by the user or caregiver at a previous time) or received during the active communication session with the sensor apparatus. Next, at step 554, any data received from the user or other device (e.g., user alert response data, calibration data, and/or user command or configuration data) are wirelessly transmitted to the implanted sensor apparatus. Per step 556, after exchange of data between the sensor apparatus and the receiver apparatus is complete, the devices are unpaired and data communication is stopped. The receiver apparatus then continues to scan for advertisement or beacon data. Examples of selectable menus for receiving user input/commands are shown in graphical presentations 610, 616, 618, 624 and 632 of FIGS. 6A and 6B. Additional graphical presentations of selectable menus for receiving user input/commands which are usable in the implanted sensor system of the present application are shown and described in U.S. patent application Ser. No. 15/472,091, previously incorporated herein.

It will be appreciated that the foregoing method 530 is merely exemplary and may include additional steps or exclude steps. It will be further appreciated that similar strategies (to those of methods 500 and 530) can be utilized for data communication with other communicative devices such as CSP 840, the partially implanted pump 1000, the fully implanted pump 1020a, and/or its associated receiver apparatus 1020. However, the specific data exchanged between the devices may tailored to the function/utility of the device communicating with the implanted sensor apparatus.

For example, in one implementation, where the communicative device is an implanted pump apparatus (or its associated receiver) and the sensor is configured to determine appropriate medicant dosage, the pump system and the implanted sensor may be brought into communication (paired) only when exchange of data is required. For example, the pump system may determine a need for and direct pairing with the sensor apparatus for transmission of pump data to the sensor, such as e.g., time-stamped medicant dispensement data/pump actuation data, medicant reservoir data, and/or data from other on-pump sensors. In another example, the sensor apparatus may determine a need for and direct pairing (via its beacon data) with the pump system for transmission of medicant dosing data (based on a pre-determined dosing algorithm or a user-specific pump operational model) or other data to the pump.

It will be recognized that while certain aspects of the disclosure are described in terms of a specific sequence of steps of a method, these descriptions are only illustrative of the broader methods of the disclosure, and may be modified as required by the particular application. Certain steps may be rendered unnecessary or optional under certain circumstances. Additionally, certain steps or functionality may be added to the disclosed embodiments, or the order of performance of two or more steps permuted. All such variations are considered to be encompassed within the disclosure disclosed and claimed herein.

While the above detailed description has shown, described, and pointed out novel features of the disclosure as applied to various embodiments, it will be understood that various omissions, substitutions, and changes in the form and details of the device or process illustrated may be made by those skilled in the art without departing from the disclosure. This description is in no way meant to be limiting, but rather should be taken as illustrative of the general principles of the disclosure. The scope of the disclosure should be determined with reference to the claims.

It will be further appreciated that while certain steps and aspects of the various methods and apparatus described herein may be performed by a human being, the disclosed aspects and individual methods and apparatus are generally computerized/computer-implemented. Computerized apparatus and methods are necessary to fully implement these aspects for any number of reasons including, without limitation, commercial viability, practicality, and even feasibility (i.e., certain steps/processes simply cannot be performed by a human being in any viable fashion).

What is claimed is:

1. Implantable sensor apparatus configured to monitor at least one physiologic parameter of a living subject, the implantable sensor apparatus comprising:
at least one sensor element;
wireless interface apparatus;
data processor apparatus in communication with each of the at least one sensor element and the wireless interface apparatus; and
data storage apparatus in data communication with the data processor apparatus, the data storage apparatus comprising at least one computer program stored thereon, the at least one computer program comprising a plurality of instructions, the plurality of instructions configured to, when executed by the data storage apparatus, cause the implantable sensor apparatus to:
collect signals from the at least one sensor element, the signals related to the physiologic parameter;
process at least a portion of the signals to generate physiologic parameter data;
determine whether to enable communication with a receiving device;
based at least in part on a determination to not enable communication, at least temporarily store the physiologic parameter data; and
based at least in part on a determination to enable communication, wirelessly transmit data configured to enable establishment of a communications session with the receiving device.

2. The implantable sensor apparatus of claim 1, wherein the implantable sensor apparatus is a fully implantable oxygen-based glucose sensor apparatus configured to monitor blood glucose concentration of the living subject.

3. The implantable sensor apparatus of claim 2, wherein the determination of whether to enable communication with the receiving device comprises at least:
determination whether data indicative of a at least one of (i) blood analyte level and/or (ii) rate of change (ROC) obtained from the physiologic parameter data is within or outside of a specified range;
based at least on part on a determination that the data indicative of the at least one blood analyte level and/or ROC is within the specified range, do not enable communication; and
based at least in part on a determination that the data indicative of the at least one blood analyte level and/or ROC is outside of the specified range, generation of data indicative of an alert condition related to the at least one blood analyte level and/or ROC of the living subject, the wireless transmission of data configured to enable establishment of the communications session with the receiving device comprising transmission of the data indicative of the alert condition.

4. The implantable sensor apparatus of claim 1, wherein the determination of whether to enable communication with the receiving device comprises at least:
determination of whether one or more criteria for calibration of the at least one sensor element are met;
based at least on part on a determination that the one or more criteria for calibration are not met, do not enable communication; and
based at least in part on a determination that at least one of the one or more criteria for calibration are met, generation of data indicative of a request for calibration, the wireless transmission of data configured to enable establishment of a communications session with the receiving device comprising transmission of the data indicative of the request for calibration.

5. The implantable sensor apparatus of claim 1, wherein the determination of whether to enable communication with the receiving device comprises at least:
determination of whether the physiologic parameter data meets or exceeds a threshold level of new data which has not been previously transmitted to the receiving device;
based at least on part on a determination that the physiologic parameter data does not meet or exceed the threshold level of new data, do not enable communication; and
based at least in part on a determination that the physiologic parameter data meets or exceeds the threshold level of new data, generation of data indicative of new physiologic parameter data, the wireless transmission of data configured to enable establishment of a communications session with the receiving device comprising transmission of the data indicative of new physiologic parameter data.

6. The implantable sensor apparatus of claim 5, wherein the plurality of instructions are configured to, when executed by the data processor apparatus, cause the implantable sensor apparatus to, after establishment of the communications session with the receiving device, transmit of at least a portion of the physiologic parameter data corresponding to new data to the receiving device via the wireless interface apparatus.

7. The implantable sensor apparatus of claim 1, wherein the wireless interface apparatus comprises a Bluetooth Low Energy (BLE)-compliant wireless interface apparatus.

8. The implantable sensor apparatus of claim 7, wherein:
the implantable sensor apparatus is configured to be implanted beneath an adipose tissue layer on a front region of a torso of the living subject; and
the BLE wireless interface apparatus is configured to transmit and receive signals through the adipose tissue layer after implantation of the implantable sensor apparatus.

9. The implantable sensor apparatus of claim 8, wherein the BLE wireless interface apparatus is further configured to broadcast beacon data on at least one channel at a plurality of regular intervals, each of the plurality of regular intervals comprising a scan window followed by a delay period.

10. The implantable sensor apparatus of claim 7, wherein:
the blue tooth low energy (BLE) wireless interface apparatus is configured to enable selective operation of the implantable sensor apparatus in a short range mode; and
the BLE wireless interface apparatus further comprises a transceiver configured to enable selective operation of the implantable sensor apparatus in a long range mode, the operation in the long range mode consuming more electrical power than the operation in the short range mode.

11. The implantable sensor apparatus of claim 10, wherein the transmission of the beacon data comprises transmission of short range beacon data via the BLE wireless interface apparatus while the implantable sensor apparatus is operated in the short range mode; and
wherein the plurality of instructions are further configured to, when executed by the data processor apparatus, cause the implantable sensor apparatus to:
determine whether, after the transmission of the short range beacon data, a predetermined time period for response has lapsed and no communications session is established with the receiving device; and
based at least in part on a determination that the response window has lapsed and no communications session is established with the receiving device, (i) enable operation of the implantable sensor apparatus in the long range mode, and
(ii) transmit transit long range beacon data.

12. A method of operating a sensor apparatus for monitoring of at least one physiologic parameter within a living subject, the sensor apparatus comprising at least one sensor element, wireless interface apparatus, processor apparatus, and storage apparatus, the method comprising:
enabling and implanting the sensor apparatus within the living subject;
operating the implanted sensor apparatus autonomously for a first period of time, the autonomously operating being independent of any other device and comprising:
collecting signals from the at least one sensor element, the signals related to the physiologic parameter;
processing at least a portion of the signals to generate physiologic parameter data; and
storing the physiologic parameter data;
determining that one or more first criteria for communication with a first receiving device are met; and
based at least in part on the determining that the one or more first criteria for communication with at least the first receiving device are met, transmitting beacon data configured to enable opportunistic wireless communication with the first receiving device.

13. The method of claim 12, wherein the determining that one or more first criteria are met for communication with the first receiving device comprises at least one of:
determining that one or more criteria for calibration of the at least one sensor element are met;
determining that the physiologic parameter data meets or exceeds a threshold level of new data which has not been previously transmitted to at least one of the first receiving device or one or more other receiving devices; and
determining that the physiologic parameter data is outside of a specified range and user notification is required.

14. The method of claim 12, wherein the first receiving device comprises a mobile computerized user device; and
the method further comprises determining one or more second criteria are met for communication with a second receiving device, the second receiving device comprising a computerized medicant delivery apparatus, the determining the one or more second criteria are met comprising determining that the physiologic parameter data is outside of a specified range and medicant delivery is required.

15. The method of claim 12, wherein:
the wireless interface apparatus comprises a blue tooth low energy (BLE) wireless interface apparatus, the BLE wireless interface apparatus configured to enable selective operation of the implantable sensor apparatus in a first range—reduced energy consumption mode; and
the transmitting beacon data comprises broadcasting, via the BLE interface, the beacon data on at least one channel at a plurality of intervals, each of the plurality of intervals comprising a scan window followed by a delay period.

16. The method of claim 15, wherein:
the wireless interface apparatus further comprises a transceiver configured to enable selective operation of the implanted sensor apparatus in a second range—high power mode, the second range greater than the first range; and
the method further comprises:
determining that a predetermined time period for response has lapsed with no establishment of a communications session with the first receiving device; and
based at least in part on the lapsed predetermined time period for response, enabling operation in the second range—high power mode for establishing a communications session with the first receiving device.

17. Implantable sensor apparatus configured to monitor at least one physiologic parameter of a living subject, the implantable sensor apparatus comprising:
at least one sensor element;
wireless interface apparatus;
data processor apparatus in data communication with each of the at least one sensor element and the wireless interface apparatus; and
data storage apparatus in data communication with the data processor apparatus, the data storage apparatus comprising at least one computer program stored thereon, the at least one computer program comprising a plurality of instructions, the plurality of instructions configured to, when executed by the data storage apparatus, cause the implantable sensor apparatus to:
operate the implanted sensor apparatus autonomously for a first period of time, the autonomous operation comprising:
collection of signals from the at least one sensor element, the signals related to the physiologic parameter;
processing of at least a portion of the signals to generate physiologic parameter data; and
at least temporarily store the physiologic parameter data;
determine at a first time that one or more criteria for enablement of communication with a receiving device are met, and based at least in part on the determination, enable establishment of a communications session with the receiving device and determine at a second time that one or more criteria for enablement of communication with a receiving device are not met, and based at least in part on the determination that the one or more criteria for enablement of communication with a receiving device are not met, continue the autonomous operation for a second period of time.

18. The implantable sensor apparatus of claim 17, wherein the determination that the one or more criteria for enablement of communication with a receiving device are met comprises at least one of:
i) identification that one or more criteria for calibration of the at least one sensor element are met;
ii) determination that the stored physiologic parameter data meets or exceeds a threshold level of new data which has not been previously transmitted to at least one of the receiving device or one or more other receiving devices;
iii) determination that the physiologic parameter data is outside of a specified range and user notification is required; and/or
iv) receipt of a request for communication from the receiving device.

19. The implantable sensor apparatus of claim 17, wherein the enablement of establishment of the communications session with the receiving device comprises:
operation of the sensor apparatus in a reduced range—low power communications mode;
evaluation of whether a predetermined response period has lapsed without establishment of communication during the operation in the reduced range—low power communications mode; and
based at least in part on lapse of the of predetermined response period without establishment of communication, operation of the sensor apparatus in an increased range—high power communications mode.
